(19) United States
(12) Patent Application Publication
(10) Pub. No.: US 2009/0317360 A1
(43) Pub. Date: Dec. 24, 2009

(54) ANTI-VIRAL INHIBITORS AND METHODS OF USE

(75) Inventors: Roopa Rai, San Carlos, CA (US); Franz Ulrich Schmitz, Mill Valley, CA (US); Christopher Don Roberts, Belmont, CA (US); Irina Slobodov, San Mateo, CA (US); Martin Robert Leivers, San Francisco, CA (US)

Correspondence Address: GlaxoSmithKline;c/o Foley & Lardner LLP, 975 Page Mill Road, Palo Alto, CA, 94304-1013, US

(73) Assignee: Genelabs Technologies, Inc.

(21) Appl. No.: 12/137,449

(22) Filed: Jun. 11, 2008

Related U.S. Application Data
(60) Provisional application No. 60/943,528, filed on Jun. 12, 2007.

(51) Int. Cl. A61K 38/21; C07D 417/14; A61K 31/541; C07D 417/12; A61K 31/5377; A61K 31/497

(52) U.S. Cl. 424/85.4; 544/58.2; 514/227.8; 544/107; 514/232.2; 544/357; 514/252.11

(57) ABSTRACT

Disclosed are compounds and compositions of Formula (I), pharmaceutically acceptable salts and solvates thereof, and their uses for treating viral infections mediated at least in part by a virus in the Flaviviridae family of viruses.

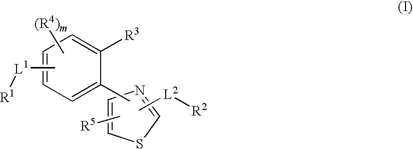

### CROSS REFERENCE TO RELATED APPLICATION

This application claims the benefit under 35 U.S.C. 119(e) to co-pending provisional application U.S. Ser. No. 60/943,528 filed on Jun. 12, 2007, which is incorporated herein by reference in its entirety.

### BACKGROUND OF THE INVENTION

### Field of the Invention

Compounds, compositions, and methods for treating viral infections in patients mediated, at least in part, by a virus in the Flaviviridae family of viruses are disclosed.

### REFERENCES

The following publications are cited in this application as superscript numbers:
1. Szabo, E. et al., Pathol. Oncol. Res. 2003, 9:215-221.
2. Hoofnagle, J. H., Hepatology 1997, 26:15 S-20S.
3. Thomson, B. J. and Finch, R. G., Clin Microbial Infect. 2005, 11:86-94.
4. Moriishi, K. and Matsuura, Y., Antivir. Chem. Chemother. 2003, 14:285-297.
5. Fried, M. W., et al. N. Engl. J Med 2002, 347:975-982.
6. Ni, Z. J. and Wagman, A. S. Curr. Opin. Drug Discov. DeveL 2004, 7, 446-459.
7. Beaulieu, P. L. and Tsantrizos, Y. S. Curr. Opin. Investig. Drugs 2004, 5, 838-850.
8. Griffith, R. C. et al., Ann. Rep. Med. Chem. 39, 223-237, 2004.
9. Watashi, K. et al., Molecular Cell, 19, 111-122, 2005
10. Horsmans, Y. et al., Hepatology, 42, 724-731, 2005

### State of the Art

Chronic infection with HCV is a major health problem associated with liver cirrhosis, hepatocellular carcinoma and liver failure. An estimated 170 million chronic carriers worldwide are at risk of developing liver disease.^{1,2} In the United States alone 2.7 million are chronically infected with HCV, and the number of HCV-related deaths in 2000 was estimated between 8,000 and 10,000, a number that is expected to increase significantly over the next years. Infection by HCV is insidious in a high proportion of chronically infected (and infectious) carriers who may not experience clinical symptoms for many years. Liver cirrhosis can ultimately lead to liver failure. Liver failure resulting from chronic HCV infection is now recognized as a leading cause of liver transplantation.

HCV is a member of the Flaviviridae family of RNA viruses that affect animals and humans. The genome is a single ˜9.6-kilobase strand of RNA, and consists of one open reading frame that encodes for a polyprotein of ˜3000 amino acids flanked by untranslated regions at both 5′ and 3′ ends (5′- and 3′-UTR). The polyprotein serves as the precursor to at least 10 separate viral proteins critical for replication and assembly of progeny viral particles. The organization of structural and non-structural proteins in the HCV polyprotein is as follows: C-E1-E2-p7-NS2-NS3-NS4a-NS4b-NS5a-NS5b. Because the replicative cycle of HCV does not involve any DNA intermediate and the virus is not integrated into the host genome, HCV infection can theoretically be cured. While the pathology of HCV infection affects mainly the liver, the virus is found in other cell types in the body including peripheral blood lymphocytes.^{3,4}

At present, the standard treatment for chronic HCV is interferon alpha (IFN-alpha) in combination with ribavirin and this requires at least six (6) months of treatment. IFN-alpha belongs to a family of naturally occurring small proteins with characteristic biological effects such as antiviral, immunoregulatory and antitumoral activities that are produced and secreted by most animal nucleated cells in response to several diseases, in particular viral infections. IFN-alpha is an important regulator of growth and differentiation affecting cellular communication and immunological control. Treatment of HCV with interferon has frequently been associated with adverse side effects such as fatigue, fever, chills, headache, myalgias, arthralgias, mild alopecia, psychiatric effects and associated disorders, autoimmune phenomena and associated disorders and thyroid dysfunction. Ribavirin, an inhibitor of inosine 5′-monophosphate dehydrogenase (IMPDH), enhances the efficacy of IFN-alpha in the treatment of HCV. Despite the introduction of ribavirin, more than 50% of the patients do not eliminate the virus with the current standard therapy of interferon-alpha (IFN) and ribavirin. By now, standard therapy of chronic hepatitis C has been changed to the combination of pegylated IFN-alpha plus ribavirin. However, a number of patients still have significant side effects, primarily related to ribavirin. Ribavirin causes significant hemolysis in 10-20% of patients treated at currently recommended doses, and the drug is both teratogenic and embryotoxic. Even with recent improvements, a substantial fraction of patients do not respond with a sustained reduction in viral load⁵ and there is a clear need for more effective antiviral therapy of HCV infection.

A number of approaches are being pursued to combat the virus. They include, for example, application of antisense oligonucleotides or ribozymes for inhibiting HCV replication. Furthermore, low-molecular weight compounds that directly inhibit HCV proteins and interfere with viral replication are considered as attractive strategies to control HCV infection. Among the viral targets, the NS3/4a protease/helicase and the NS5b RNA-dependent RNA polymerase are considered the most promising viral targets for new drugs.⁶⁻⁸

Besides targeting viral genes and their transcription and translation products, antiviral activity can also be achieved by targeting host cell proteins that are necessary for viral replication. For example, Watashi et al.⁹ show how antiviral activity can be achieved by inhibiting host cell cyclophilins. Alternatively, a potent TLR7 agonist has been shown to reduce HCV plasma levels in humans.¹⁰

However, none of the compounds described above have progressed beyond clinical trials.^{6,8}

In view of the worldwide epidemic level of HCV and other members of the Flaviviridae family of viruses, and further in view of the limited treatment options, there is a strong need for new effective drugs for treating infections cause by these viruses.

### SUMMARY OF THE INVENTION

In one embodiment, the present invention provides a compound that is Formula (I):

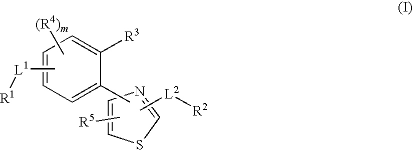

or a pharmaceutically acceptable salt or solvate thereof,
wherein:

L¹ and L² are independently selected from the group consisting of —C(O)NR^{a}-T-, —NR^{a}—C(O)-T-, —NR^{a}C(O)NR^{a}-T-, —NR^{a}C(O)C(O)-T-, —NR^{a}C(O)O-T-, —CH₂NR^{a}-T-, —NR^{a}CH₂-T-, —S(O)₂NH-T-, —NHS(O)₂-T-, and —CH₂NHS(O)₂-T-, where T is attached to R¹ or R² and is independently a covalent bond or C₁₋₃alkylene;

R^{a} is independently hydrogen or alkyl;

R¹ and R² are independently selected from the group consisting of alkyl, substituted alkyl, cycloalkyl, substituted cycloalkyl, heterocyclic, substituted heterocyclic, aryl, substituted aryl, heteroaryl, and substituted heteroaryl;

R³ and R⁵ are independently selected from the group consisting of hydrogen, halo, alkyl, substituted alkyl, alkenyl, substituted alkenyl, alkynyl, substituted alkynyl, hydroxy, alkoxy, substituted alkoxy, amino, substituted amino, azido, cycloalkyl, substituted cycloalkyl, and cyano;

each R⁴ is independently selected from the group consisting of halo, alkyl, substituted alkyl, alkoxy, substituted alkoxy, and hydroxy; and

m is 0, 1, 2 or 3.

In one embodiment provided is a pharmaceutical composition comprising a pharmaceutically acceptable carrier and a therapeutically effective amount of a compound of Formula (I).

In one embodiment provided is a method for treating a viral infection in a patient mediated at least in part by a virus in the Flaviviridae family of viruses, comprising administering to said patient a composition of Formula (I). In some aspects, the viral infection is mediated by hepatitis C virus.

These and other embodiments of the invention are further described in the text that follows.

### DETAILED DESCRIPTION OF THE INVENTION

Throughout this application, references are made to various embodiments relating to compounds, compositions, and methods. The various embodiments described are meant to provide a variety illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

### Definitions

It is to be understood that the terminology used herein is for the purpose of describing particular embodiments only and is not intended to limit the scope of the present invention. In this specification and in the claims that follow, reference will be made to a number of terms that shall be defined to have the following meanings:

“Alkyl” refers to monovalent saturated aliphatic hydrocarbyl groups having from 1 to 10 carbon atoms and, in some embodiments, from 1 to 6 carbon atoms. “C_{x-y}alkyl” refers to alkyl groups having from x to y carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Substituted alkyl” refers to an alkyl group having from 1 to 5 and, in some embodiments, 1 to 3 or 1 to 2 substituents selected from the group consisting of alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, spirocycloalkyl, SO₃H, substituted sulfonyl, sulfonyloxy, thioacyl, thiocyanate, thiol, alkylthio, and substituted alkylthio, wherein said substituents are as defined herein.

“Alkylidene” or “alkylene” refers to divalent saturated aliphatic hydrocarbyl groups having from 1 to 10 carbon atoms and, in some embodiments, from 1 to 6 carbon atoms. “(Cᵤ₋ᵥ)alkylene” refers to alkylene groups having from u to v carbon atoms. The alkylidene and alkylene groups include branched and straight chain hydrocarbyl groups. For example “(C₁₋₆)alkylene” is meant to include methylene, ethylene, propylene, 2-methypropylene, pentylene, and the like.

“Substituted alkylidene” or “substituted alkylene” refers to an alkylidene group having from 1 to 5 and, in some embodiments, 1 to 3 or 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, oxo, thione, spirocycloalkyl, SO₃H, substituted sulfonyl, sulfonyloxy, thioacyl, thiocyanate, thiol, alkylthio, and substituted alkylthio, wherein said substituents are as defined herein.

“Alkenyl” refers to a linear or branched hydrocarbyl group having from 2 to 10 carbon atoms and in some embodiments from 2 to 6 carbon atoms or 2 to 4 carbon atoms and having at least 1 site of vinyl unsaturation (>C═C<). For example, (C_{x-Cy})alkenyl refers to alkenyl groups having from x to y carbon atoms and is meant to include for example, ethenyl, propenyl, 1,3-butadienyl, and the like.

“Substituted alkenyl” refers to alkenyl groups having from 1 to 3 substituents and, in some embodiments, 1 to 2 substituents selected from the group consisting of alkoxy, substituted alkoxy, acyl, acylamino, acyloxy, alkyl, substituted alkyl, alkynyl, substituted alkynyl, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to a vinyl (unsaturated) carbon atom.

“Alkynyl” refers to a linear monovalent hydrocarbon radical or a branched monovalent hydrocarbon radical containing at least one triple bond. The term “alkynyl” is also meant to include those hydrocarbyl groups having one triple bond and one double bond. For example, (C₂-C₆)alkynyl is meant to include ethynyl, propynyl, and the like.

“Substituted alkynyl” refers to alkynyl groups having from 1 to 3 substituents and, in some embodiments, from 1 to 2 substituents selected from the group consisting of alkoxy, substituted alkoxy, acyl, acylamino, acyloxy, alkyl, substituted alkyl, alkenyl, substituted alkenyl, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are as defined herein and with the proviso that any hydroxy or thiol substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is as defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, substituted hydrazino-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, aryl, substituted aryl, substituted hydrazino, heteroaryl, substituted heteroaryl, heterocyclic, and substituted heterocyclic are as defined herein. Acyl includes the “acetyl” group CH₃C(O)—.

“Acylamino” refers to the groups —NR²⁰C(O)alkyl, —NR²⁰C(O)substituted alkyl, —NR²⁰C(O)cycloalkyl, —NR²⁰C(O)substituted cycloalkyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic wherein R²⁰ is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amino” refers to the group —NH₂.

“Substituted amino” refers to the group —NR²¹R²² where R²¹ and R²² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²¹ and R²² are optionally joined together with the nitrogen bound thereto to form a heterocyclic or substituted heterocyclic group, provided that R²¹ and R²² are both not hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein. When R²¹ is hydrogen and R²² is alkyl, the substituted amino group is sometimes referred to herein as alkylamino. When R²¹ and R²² are alkyl, the substituted amino group is sometimes referred to herein as dialkylamino. When referring to a monosubstituted amino, it is meant that either R²¹ or R²² is hydrogen but not both. When referring to a disubstituted amino, it is meant that neither R²¹ nor R²² are hydrogen.

“Hydroxyamino” refers to the group —NHOH.

“Alkoxyamino” refers to the group —NHO-alkyl wherein alkyl is defined herein.

“Aminocarbonyl” refers to the group —C(O)NR²³R²⁴ where R²¹ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, hydroxy, alkoxy, substituted alkoxy, amino, substituted amino, and acylamino, and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminothiocarbonyl” refers to the group —C(S)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminocarbonylamino” refers to the group —NR²⁰C(O)NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminothiocarbonylamino” refers to the group —NR²⁰C(S)NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminocarbonyloxy” refers to the group —O—C(O)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonyl” refers to the group —SO₂NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonyloxy” refers to the group —O—SO₂NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonylamino” refers to the group —NR²⁰—SO₂NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amidino” refers to the group —C(═NR²⁵)NR²³R²⁴ where R²⁵, R²³, and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aryl” or “Ar” refers to an aromatic group of from 6 to 14 carbon atoms and no ring heteroatoms and having a single ring (e.g., phenyl) or multiple condensed (fused) rings (e.g., naphthyl or anthryl). For multiple ring systems, including fused, bridged, and spiro ring systems having aromatic and non-aromatic rings that have no ring heteroatoms, the term “Aryl” or “Ar” applies when the point of attachment is at an aromatic carbon atom (e.g., 5,6,7,8 tetrahydronaphthalene-2-yl is an aryl group as its point of attachment is at the 2-position of the aromatic phenyl ring).

“Substituted aryl” refers to aryl groups which are substituted with 1 to 8 and, in some embodiments, 1 to 5, 1 to 3, or 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are defined herein.

“Aryloxy” refers to the group —O-aryl, where aryl is as defined herein, that includes, by way of example, phenoxy and naphthyloxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Azido” refers to the group —N₃.

“Hydrazino” refers to the group —NHNH₂.

“Substituted hydrazino” refers to the group —NR²⁶NR²⁷R²⁸ where R²⁶, R²⁷, and R²⁸ are independently selected from the group consisting of hydrogen, alkyl, substituted alkyl, alkenyl, substituted alkenyl, alkynyl, substituted alkynyl, aryl, substituted aryl, carboxyl ester, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²⁷ and R²⁸ are optionally joined, together with the nitrogen bound thereto to form a heterocyclic or substituted heterocyclic group, provided that R²⁷ and R²⁸ are both not hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cyano” or “carbonitrile” refers to the group —CN.

“Carbonyl” refers to the divalent group —C(O)— which is equivalent to —C(═O)—.

“Carboxyl” or “carboxy” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)amino” refers to the group —NR²⁰—C(O)O-alkyl, —NR²⁰—C(O)O-substituted alkyl, —NR²⁰—C(O)O-alkenyl, —NR²⁰—C(O)O-substituted alkenyl, —NR²⁰—C(O)O-alkynyl, —NR²⁰—C(O)O-substituted alkynyl, —NR²⁰—C(O)O-aryl, —NR²⁰—C(O)O-substituted aryl, —NR²⁰—C(O)O-cycloalkyl, —NR²⁰—C(O)O-substituted cycloalkyl, —NR²⁰—C(O)O-heteroaryl, —NR²⁰—C(O)O-substituted heteroaryl, —NR²⁰—C(O)O-heterocyclic, and —NR²⁰—C(O)O-substituted heterocyclic wherein R²⁰ is alkyl or hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cycloalkyl” refers to a saturated or partially saturated cyclic group of from 3 to 14 carbon atoms and no ring heteroatoms and having a single ring or multiple rings including fused, bridged, and spiro ring systems. For multiple ring systems having aromatic and non-aromatic rings that have no ring heteroatoms, the term “cycloalkyl” applies when the point of attachment is at a non-aromatic carbon atom (e.g. 5,6,7,8,-tetrahydronaphthalene-5-yl). The term “Cycloalkyl” includes cycloalkenyl groups. Examples of cycloalkyl groups include, for instance, adamantyl, cyclopropyl, cyclobutyl, cyclopentyl, cyclooctyl, and cyclohexenyl. “Cᵤ₋ᵥcycloalkyl” refers to cycloalkyl groups having u to v carbon atoms.

“Cycloalkenyl” refers to a partially saturated cycloalkyl ring having at least one site of >C═C<ring unsaturation.

“Substituted cycloalkyl” refers to a cycloalkyl group, as defined herein, having from 1 to 8, or 1 to 5, or in some embodiments 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are as defined herein. The term “substituted cycloalkyl” includes substituted cycloalkenyl groups.

“Cycloalkyloxy” refers to —O-cycloalkyl wherein cycloalkyl is as defined herein.

“Substituted cycloalkyloxy refers to —O-(substituted cycloalkyl) wherein substituted cycloalkyl is as defined herein.

“Cycloalkylthio” refers to —S-cycloalkyl wherein cycloalkyl is as defined herein.

“Substituted cycloalkylthio” refers to —S-(substituted cycloalkyl).

“Guanidino” refers to the group —NHC(═NH)NH₂.

“Substituted guanidino” refers to —NR²⁹C(═NR²⁹)N(R²⁹)₂ where each R²⁹ is independently selected from the group consisting of hydrogen, alkyl, substituted alkyl, aryl, substituted aryl, heteroaryl, substituted heteroaryl, heterocyclyl, and substituted heterocyclyl and two R²⁹ groups attached to a common guanidino nitrogen atom are optionally joined together with the nitrogen bound thereto to form a heterocyclic or substituted heterocyclic group, provided that at least one R²⁹ is not hydrogen, and wherein said substituents are as defined herein.

“Halo” or “halogen” refers to fluoro, chloro, bromo, and iodo.

“Haloalkyl” refers to substitution of alkyl groups with 1 to 5 or in some embodiments 1 to 3 halo groups.

“Haloalkoxy” refers to substitution of alkoxy groups with 1 to 5 or in some embodiments 1 to 3 halo groups.

“Hydroxy” or “hydroxyl” refers to the group —OH.

“Heteroaryl” refers to an aromatic group of from 1 to 14 carbon atoms and 1 to 6 heteroatoms selected from the group consisting of oxygen, nitrogen, and sulfur and includes single ring (e.g. imidazolyl) and multiple ring systems (e.g. benzimidazol-2-yl and benzimidazol-6-yl). For multiple ring systems, including fused, bridged, and spiro ring systems having aromatic and non-aromatic rings, the term “heteroaryl” applies if there is at least one ring heteroatom and the point of attachment is at an atom of an aromatic ring (e.g. 1,2,3,4-tetrahydroquinolin-6-yl and 5,6,7,8-tetrahydroquinolin-3-yl). In one embodiment, the nitrogen and/or the sulfur ring atom(s) of the heteroaryl group are optionally oxidized to provide for the N-oxide (N→O), sulfinyl, or sulfonyl moieties. More specifically the term heteroaryl includes, but is not limited to, pyridyl, furanyl, thienyl, thiazolyl, isothiazolyl, triazolyl, imidazolyl, isoxazolyl, pyrrolyl, pyrazolyl, pyridazinyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, quinazolinonyl, benzimidazolyl, benzisoxazolyl, or benzothienyl.

“Substituted heteroaryl” refers to heteroaryl groups that are substituted with from 1 to 8 or in some embodiments 1 to 5, or 1 to 3, or 1 to 2 substituents selected from the group consisting of the substituents defined for substituted aryl.

“Heteroaryloxy” refers to —O-heteroaryl wherein heteroaryl is as defined herein.

“Substituted heteroaryloxy refers to the group —O-(substituted heteroaryl) wherein substituted heteroaryl is as defined herein.

“Heteroarylthio” refers to the group —S-heteroaryl wherein heteroaryl is as defined herein.

“Substituted heteroarylthio” refers to the group —S-(substituted heteroaryl) wherein substituted heteroaryl is as defined herein.

“Heterocyclic” or “heterocycle” or “heterocycloalkyl” or “heterocyclyl” refers to a saturated or partially saturated cyclic group having from 1 to 14 carbon atoms and from 1 to 6 heteroatoms selected from the group consisting of nitrogen, sulfur, or oxygen and includes single ring and multiple ring systems including fused, bridged, and spiro ring systems. For multiple ring systems having aromatic and/or non-aromatic rings, the terms “heterocyclic”, “heterocycle”, “heterocycloalkyl”, or “heterocyclyl” apply when there is at least one ring heteroatom and the point of attachment is at an atom of a non-aromatic ring (e.g. 1,2,3,4-tetrahydroquinoline-3-yl, 5,6,7,8-tetrahydroquinoline-6-yl, and decahydroquinolin-6-yl). In one embodiment, the nitrogen and/or sulfur atom(s) of the heterocyclic group are optionally oxidized to provide for the N-oxide, sulfinyl, sulfonyl moieties. More specifically the heterocyclyl includes, but is not limited to, tetrahydropyranyl, piperidinyl, N-methylpiperidin-3-yl, piperazinyl, N-methylpyrrolidin-3-yl, 3-pyrrolidinyl, 2-pyrrolidon-1-yl, morpholinyl, and pyrrolidinyl. A prefix indicating the number of carbon atoms (e.g., C₃-C₁₀) refers to the total number of carbon atoms in the portion of the heterocyclyl group exclusive of the number of heteroatoms.

“Substituted heterocyclic” or “Substituted heterocycle” or “substituted heterocycloalkyl” or “substituted heterocyclyl” refers to heterocyclic groups, as defined herein, that are substituted with from 1 to 5 or in some embodiments 1 to 3 of the substituents as defined for substituted cycloalkyl.

“Heterocyclyloxy” refers to the group —O-heterocycyl wherein heterocyclyl is as defined herein.

“Substituted heterocyclyloxy” refers to the group —O-(substituted heterocycyl) wherein substituted heterocyclyl is as defined herein.

“Heterocyclylthio” refers to the group —S-heterocycyl wherein heterocyclyl is as defined herein.

“Substituted heterocyclylthio” refers to the group —S-(substituted heterocycyl) wherein substituted heterocyclyl is as defined herein.

Examples of heterocycle and heteroaryl groups include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O).

“Oxide” refers to products resulting from the oxidation of one or more heteroatoms. Examples include N-oxides, sulfoxides, and sulfones.

“Spirocycloalkyl” refers to a 3 to 10 member cyclic substituent formed by replacement of two hydrogen atoms at a common carbon atom with an alkylene group having 2 to 9 carbon atoms, as exemplified by the following structure wherein the methylene group shown here attached to bonds marked with wavy lines is substituted with a spirocycloalkyl group:

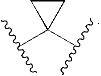

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-alkynyl, —SO₂-substituted alkynyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein. Substituted sulfonyl includes groups such as methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cylcoalkyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thiol” refers to the group —SH.

“Alkylthio” refers to the group —S-alkyl wherein alkyl is as defined herein.

“Substituted alkylthio” refers to the group —S-(substituted alkyl) wherein substituted alkyl is as defined herein.

“Thiocarbonyl” refers to the divalent group —C(S)— which is equivalent to —C(═S)—.

“Thione” refers to the atom (═S).

“Thiocyanate” refers to the group —SCN.

“Compound” and “compounds” as used herein refers to a compound encompassed by the generic formulae disclosed herein, any subgenus of those generic formulae, and any forms of the compounds within the generic and subgeneric formulae, including the racemates, stereoisomers, and tautomers of the compound or compounds.

“Racemates” refers to a mixture of enantiomers.

“Solvate” or “solvates” of a compound refer to those compounds, where compounds is as defined above, that are bound to a stoichiometric or non-stoichiometric amount of a solvent. Solvates of a compound includes solvates of all forms of the compound. Preferred solvents are volatile, non-toxic, and/or acceptable for administration to humans in trace amounts. Suitable solvates include water.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

“Patient” refers to mammals and includes humans and non-human mammals.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

Unless indicated otherwise, the nomenclature of substituents that are not explicitly defined herein are arrived at by naming the terminal portion of the functionality followed by the adjacent functionality toward the point of attachment. For example, the substituent “arylalkyloxycabonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

Accordingly in one embodiment, provided is a compound that is Formula (I):

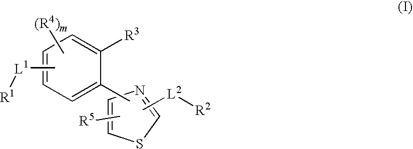

or a pharmaceutically acceptable salt or solvate thereof, wherein:

L¹ and L² are independently selected from the group consisting of —C(O)NR^{a}-T-, —NR^{a}—C(O)-T-, —NR^{a}C(O)NR^{a}-T-, —NR^{a}C(O)C(O)-T-, —NR^{a}C(O)O-T-, —CH₂NR^{a}-T-, —NR^{a}CH₂-T-, —S(O)₂NH-T-, —NHS(O)₂-T-, and —CH₂NHS(O)₂-T-, where T is attached to R¹ or R² and is independently a covalent bond or C₁₋₃alkylene;

R^{a} is independently hydrogen or alkyl;

R¹ and R² are independently selected from the group consisting of alkyl, substituted alkyl, cycloalkyl, substituted cycloalkyl, heterocyclic, substituted heterocyclic, aryl, substituted aryl, heteroaryl, and substituted heteroaryl;

R³ and R⁵ are independently selected from the group consisting of hydrogen, halo, alkyl, substituted alkyl, alkenyl, substituted alkenyl, alkynyl, substituted alkynyl, hydroxy, alkoxy, substituted alkoxy, amino, substituted amino, azido, cycloalkyl, substituted cycloalkyl, and cyano;

each R⁴ is independently selected from the group consisting of halo, alkyl, substituted alkyl, alkoxy, substituted alkoxy, and hydroxy; and

m is 0, 1, 2 or 3.

In one embodiment, provided is a compound of Formula (I) that is a pharmaceutically acceptable salt.

In one embodiment, provided is a compound of Formula (I) that is a solvate. In some aspects, the solvate is a solvate of a pharmaceutically acceptable salt of Formula (I).

In one embodiment, provided is a compound that is Formula (Ia) or (Ib)

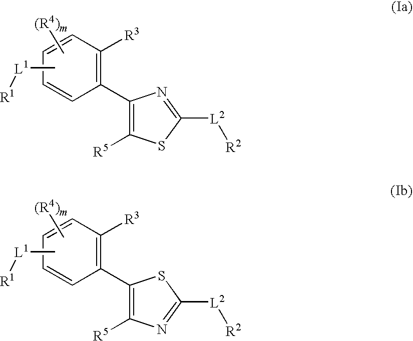

or a pharmaceutically acceptable salt or solvate thereof, wherein L¹, L², R¹, R², R³, R⁴, R¹⁵, and m are as defined for Formula (I).

In one embodiment, provided is a compound that is Formula (Ic) or (Id)

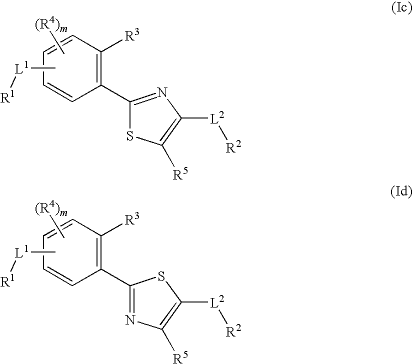

or a pharmaceutically acceptable salt or solvate thereof, wherein L², L¹, R¹, R², R³, R⁴, R⁵, and m are as defined for Formula (I).

In other embodiments, provided is a compound of Formula (Ia′)-(Id′) or a pharmaceutically acceptable salt or solvate thereof wherein L¹ is para to the thiazole ring and L¹, L², R¹, R², R³, R⁴, R⁵, and m are as defined for Formula (I):

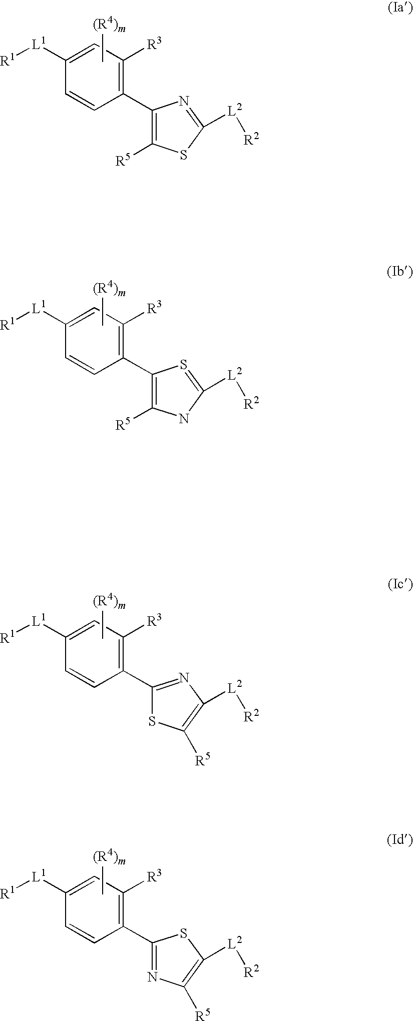

In other embodiments, provided is a compound of Formula (Ia″)-(Id″) or a pharmaceutically acceptable salt or solvate thereof wherein L¹ is para to R³ and L¹, L², R¹, R², R³, R⁴, R⁵, and m are as defined for Formula (I):

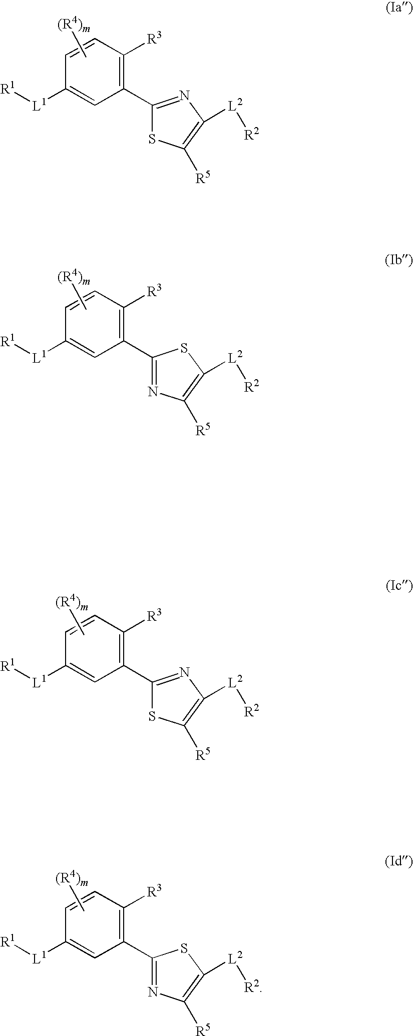

Various embodiments relating to a compound of Formula (I), (Ia)-(Id), (Ia′)-(Id′), and (Ia″)-(Id″) and a pharmaceutically acceptable salt or solvate thereof are given below. These embodiments when referring to different substituents or variables can be combined with each other or with any other embodiments described in this application. In some aspects, provided are compounds of Formula (I), (Ia)-(Id), (Ia′)-(Id′), and (Ia″)-(Id″) having one or more of the following features below.

In some embodiments, L¹ and L² are independently —C(O)NR^{a}-T- or —NR^{a}—C(O)-T-.

In some embodiments, R³ is hydrogen, halo, alkoxy, or haloalkoxy.

In some embodiments, R⁴ is alkyl.

In some embodiments, R¹ and R² are independently selected from the group consisting of cycloalkyl, substituted cycloalkyl, heterocyclic, substituted heterocyclic, aryl, substituted aryl, heteroaryl, and substituted heteroaryl.

In some embodiments, R¹ are R² are substituted phenyl or substituted herteroaryl.

In some embodiements, R¹ and R² are identical.

In some embodiments, R¹ and R² are independently -G-(Z)ₚ, wherein G is selected from the group consisting of alkyl, alkoxy, amino, acyl,

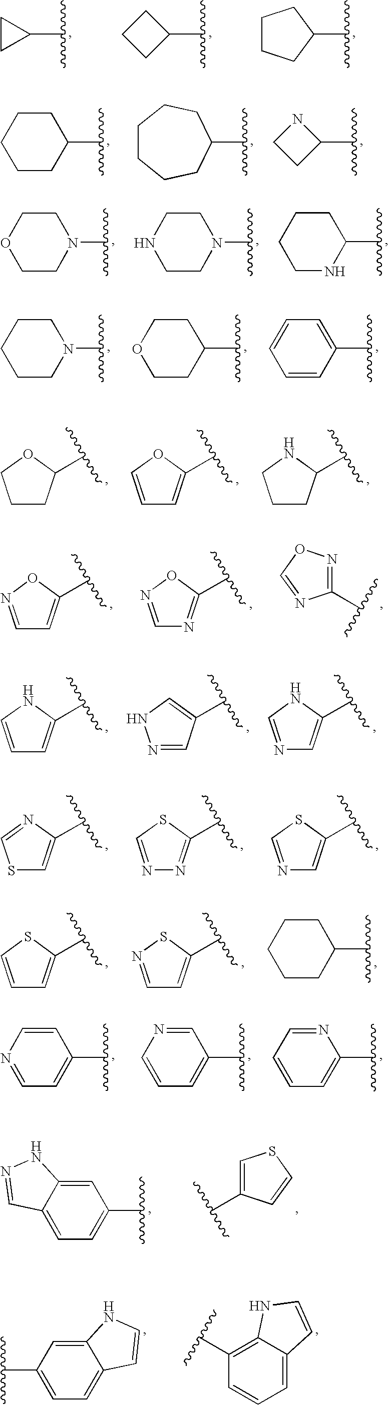

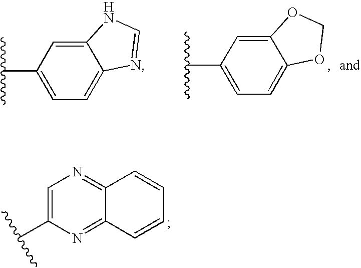

Z is independently selected from the group consisting of alkyl, substituted alkyl, amino, substituted amino, acyl, cyano, halo, hydroxy, alkoxy, substituted alkoxy, cycloalkyl, substituted cycloalkyl, heterocyclic, substituted heterocyclic, aryl, substituted aryl, heteroaryl, substituted heteroaryl, aminocarbonyl, and acylamino; and

p is 0, 1, 2, or 3.

In some embodiments, R¹ and R² are independently -G-(Z)ₚ and p is 0.

In some embodiments, Z is independently selected from the group consisting of —F, Cl, —Br, —CH₃, —CF₃, —OMe, —OCF₃, —CN, carboxyl ester, —NHC(O)CH₃,

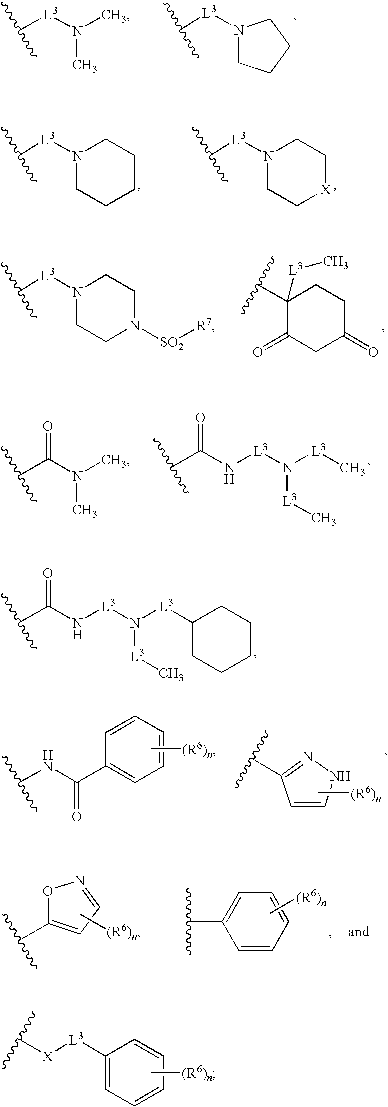

wherein

each R⁶ is independently selected from the group consisting of alkyl, substituted alkyl, alkoxy, substituted alkoxy, and halo;

R⁷ is alkyl, substituted alkyl, cyloalkyl, substituted cycloalkyl, amino, or substituted amino;

R⁸ is hydrogen, alkyl, or substituted alkyl,

L³ is a covalent bond or is C₁₋₃alkylene;

X is selected from the group consisting of O, S, S(O), S(O)₂, and NR⁸; and

n is 0, 1, 2, or 3. In some aspects, G is phenyl.

In some embodiments, Z is independently selected from the group consisting of —F, —Cl, —Br, —CH₃, —CF₃, CN, hydroxy, alkoxy,

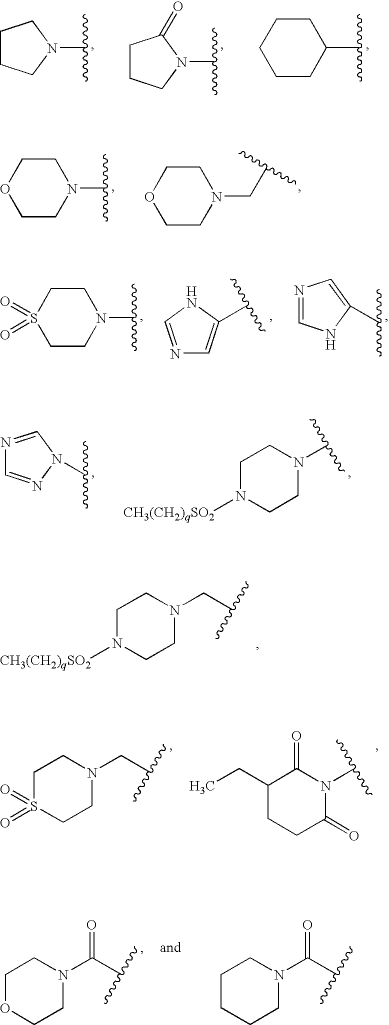

where q is 0, 1, 2, or 3.

In some embodiments, R¹ is selected from the group consisting of

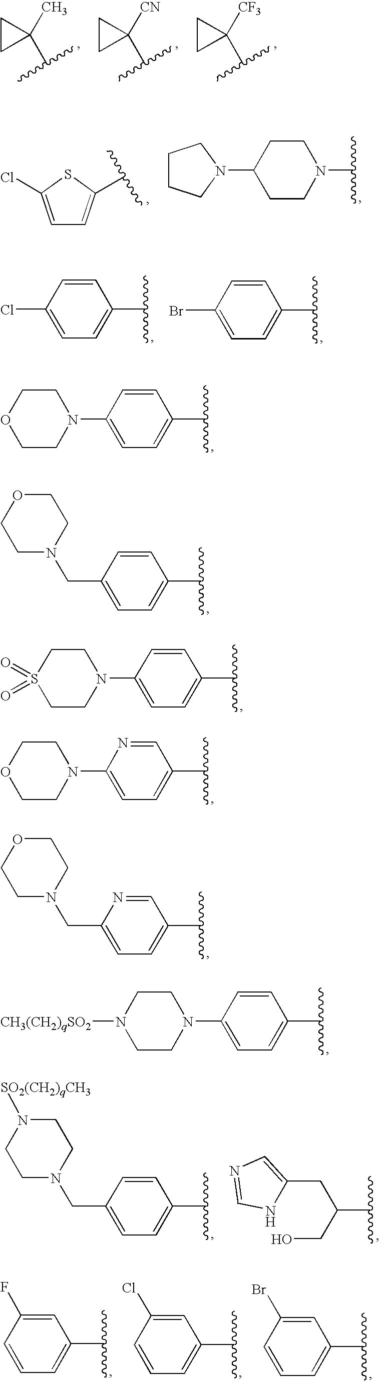

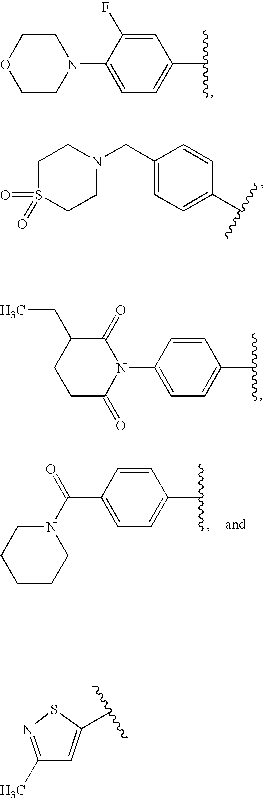

where q is 0, 1, 2, or 3. In some aspects, G is phenyl.

In some embodiments, R² is selected from the group consisting of

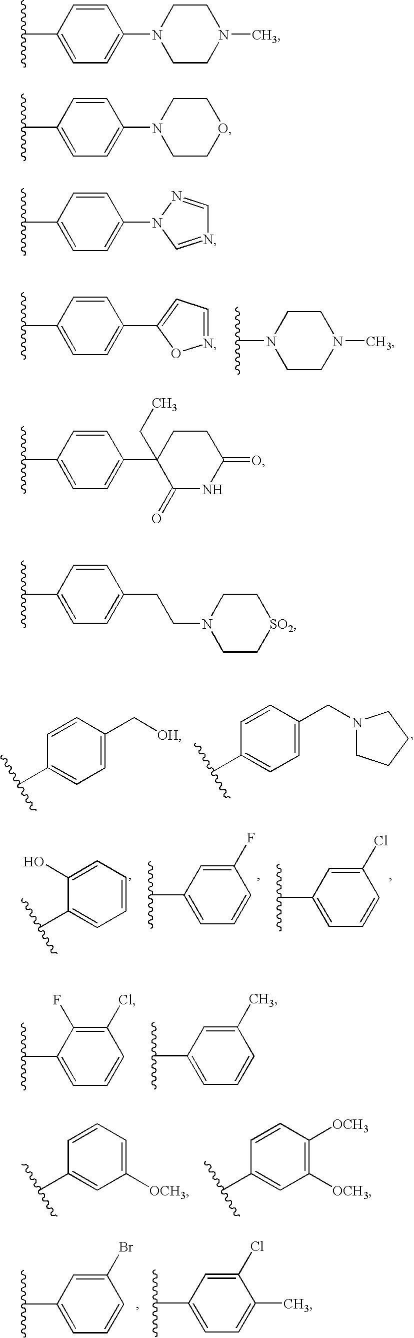

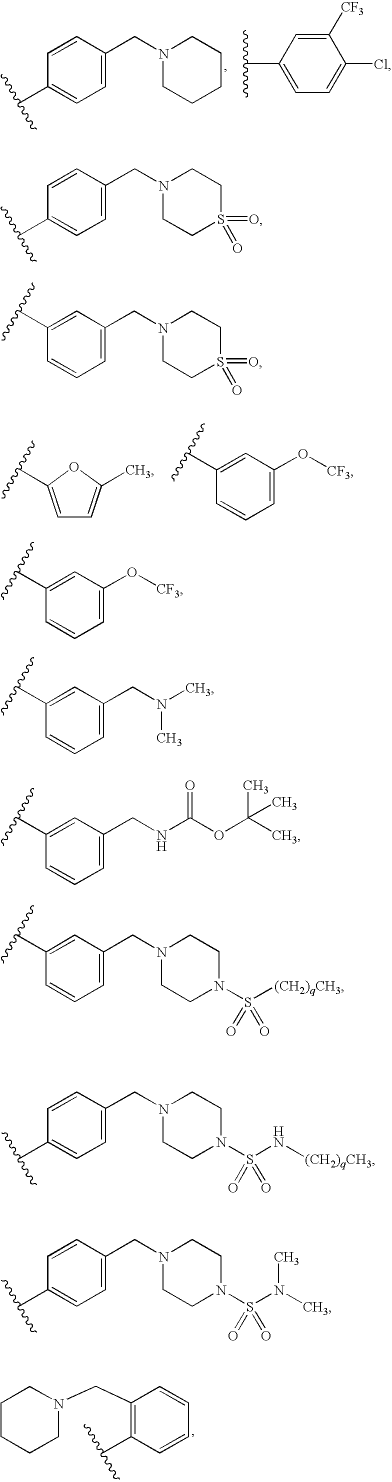

and where q is 0, 1, 2, or 3.

In yet other embodiments, the present invention provides a compound selected from Table 1 or a pharmaceutically acceptable salt thereof.

| Cmpd. # | Structure | Name |
|---|---|---|
| 101 | 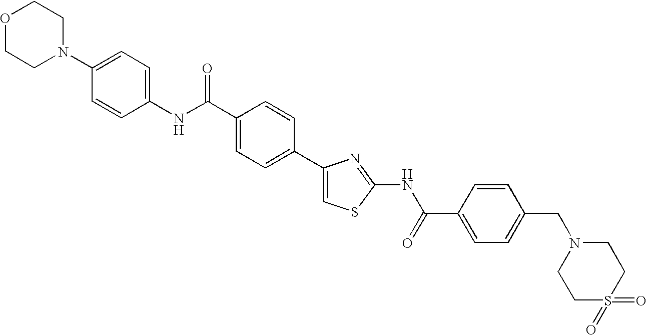 | 4-{2-[4-(1,1-Dioxo- thiomorpholin-4- ylmethyl)- benzoylamino]- thiazol-4-yl}-N-(4- morpholin-4-yl- phenyl)-benzamide |
| 102 | 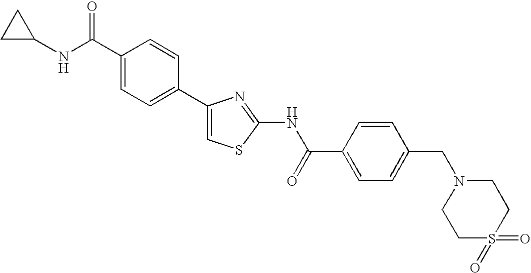 | 4-{2-[4-(1,1-Dioxo- thiomorpholin-4- ylmethyl)- benzoylamino]- thiazol-4-yl}-N- cyclopropyl- benzamide |
| 103 | 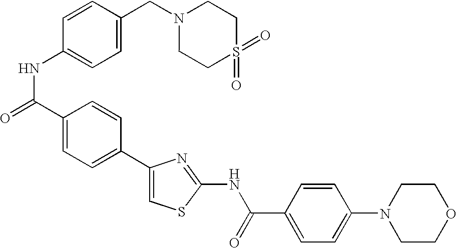 | 4-[2-(4-Morpholin-4- yl-benzoylamino)- thiazol-4-yl]-N-[4- (1,1-dioxo- thiomorpholin-4- ylmethyl)-phenyl]- benzamide |
| 104 | 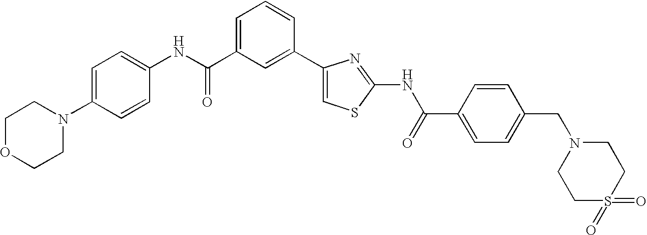 | 3-{2-[4-(1,1-Dioxo- thiomorpholin-4- ylmethyl)- benzoylamino]- thiazol-4-yl}-N-(4- morpholin-4-yl- phenyl)-benzamide |
| 105 | 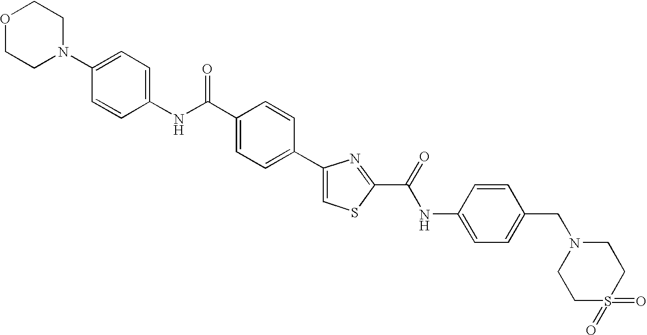 | 4-[4-(4-Morpholin-4- yl-phenylcarbamoyl)- phenyl]-thiazole-2- carboxylic acid [4- (1,1-dioxo- thiomorpholin-4- ylmethyl)-phenyl]- amide |
| 106 | 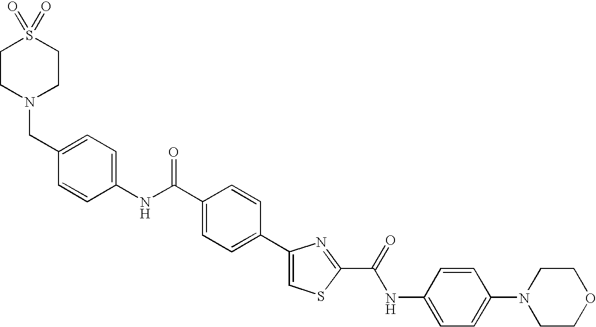 | 4-{4-[4-(1,1-Dioxo- thiomorpholin-4- ylmethyl)- phenylcarbamoyl]- phenyl}-thiazole-2- carboxylic acid (4- morpholin-4-yl- phenyl)-amide |
| 107 | 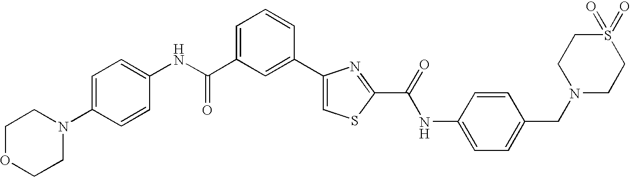 | 4-[3-(4-Morpholin-4- yl-phenylcarbamoyl)- phenyl]-thiazole-2- carboxylic acid [4- (1,1-dioxo- thiomorpholin-4- ylmethyl)-phenyl]- amide |
| 108 | 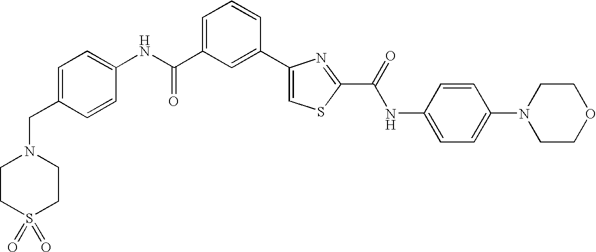 | 4-{3-[4-(1,1-Dioxo- thiomorpholin-4- ylmethyl)- phenylcarbamoyl]- phenyl}-thiazole-2- carboxylic acid (4- morpholin-4-yl- phenyl)-amide |
| 109 | 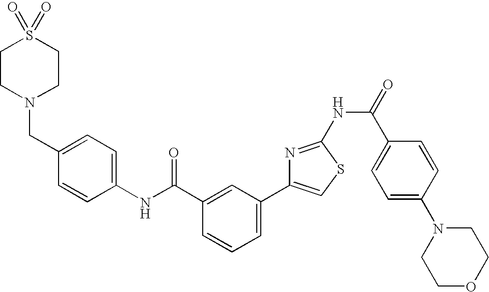 | 3-[2-(4-Morpholin-4- yl-benzoylamino)- thiazol-4-yl]-N-[4- (1,1-dioxo- thiomorpholin-4- ylmethyl)-phenyl]- benzamide |
| 110 | 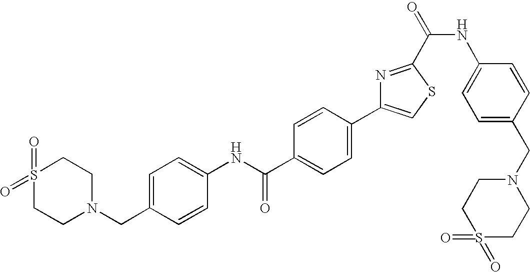 | 4-{4-[4-(1,1-Dioxo- thiomorpholin-4- ylmethyl)- phenylcarbamoyl]- phenyl}-thiazole-2- carboxylic acid [4- (1,1-dioxo- thiomorpholin-4- ylmethyl)-phenyl]- amide |
| 111 | 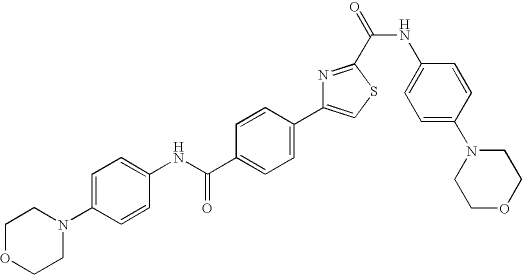 | 4-[4-(4-Morpholin-4- yl-phenylcarbamoyl)- phenyl]-thiazole-2- carboxylic acid (4- morpholin-4-yl- phenyl)-amide |
| 112 | 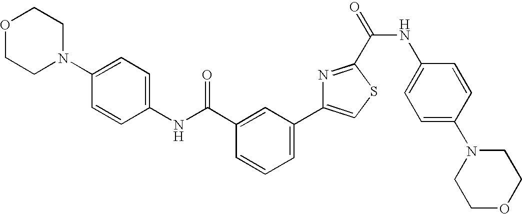 | 4-[3-(4-Morpholin-4- yl-phenylcarbamoyl)- phenyl]-thiazole-2- carboxylic acid (4- morpholin-4-yl- phenyl)-amide |
| 113 | 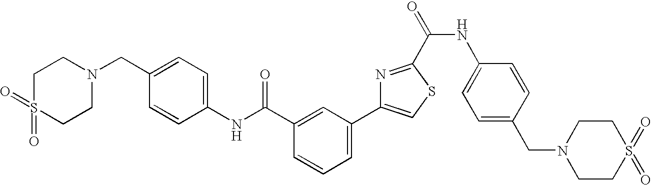 | 4-{3-[4-(1,1-Dioxo- thiomorpholin-4- ylmethyl)- phenylcarbamoyl]- phenyl}-thiazole-2- carboxylic acid [4- (1,1-dioxo- thiomorpholin-4- ylmethyl)-phenyl]- amide |
| 114 | 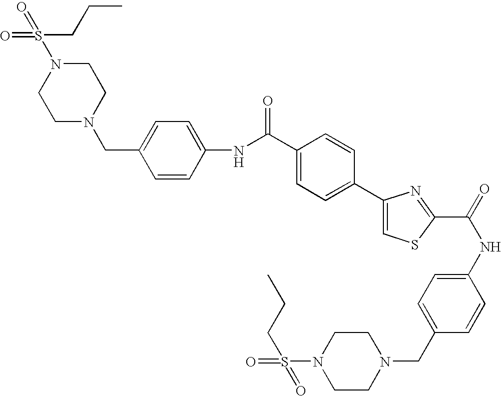 | 4-(4-{4-[4-(Propane- 1-sulfonyl)- piperazin-1- ylmethyl]- phenylcarbamoyl}- phenyl)-thiazole-2- carboxylic acid {4- [4-(propane-1- sulfonyl)-piperazin- 1-ylmethyl]-phenyl}- amide |
| 115 | 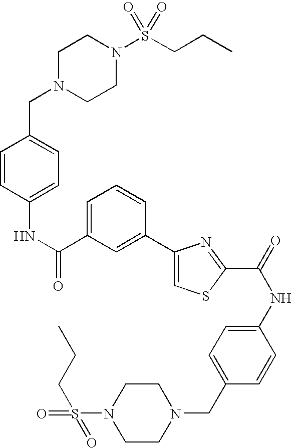 | 4-(3-{4-[4-(Propane- 1-sulfonyl)- piperazin-1- ylmethyl]- phenylcarbamoyl}- phenyl)-thiazole-2- carboxylic acid {4- [4-(propane-1- sulfonyl)-piperazin- 1-ylmethyl]-phenyl}- amide |
| 116 | 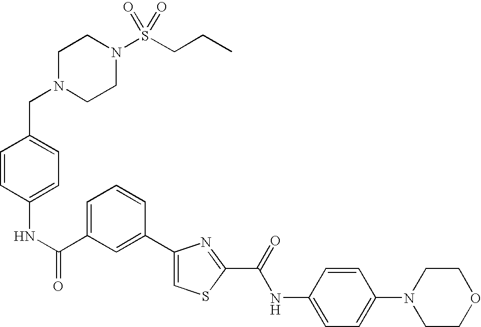 | 4-(3-{4-[4-(Propane- 1-sulfonyl)- piperazin-1- ylmethyl]- phenylcarbamoyl}- phenyl)-thiazole-2- carboxylic acid (4- morpholin-4-yl- phenyl)-amide |

In other embodiments, provided are pharmaceutical compositions comprising a pharmaceutically acceptable diluent and a therapeutically effective amount of one of the compounds described herein or mixtures of one or more of such compounds.

In other embodiments, provided are methods for treating a viral infection mediated at least in part by a virus in the Flaviviridae family of viruses, such as HCV, in patients which methods comprise administering to a patient, that has been diagnosed with said viral infection or is at risk of developing said viral infection, a pharmaceutical composition comprising a pharmaceutically acceptable diluent and a therapeutically effective amount of one of the compounds described herein or mixtures of one or more of such compounds. In another aspect, present provided are use of the compounds of Formula (I) for the preparation of a medicament for treating or preventing said infections. In other aspects the patient is a human.

In yet another embodiment provided are methods of treating or preventing viral infections in patients in combination with the administration of a therapeutically effective amount of one or more agents active against HCV. Active agents against HCV include ribavirin, levovirin, viramidine, thymosin alpha-1, an inhibitor of NS3 serine protease, and inhibitor of inosine monophosphate dehydrogenase, interferon-alpha, pegylated interferon-alpha, alone or in combination with ribavirin or viramidine. In one example, the additional agent active against HCV is interferon-alpha or pegylated interferon-alpha alone or in combination with ribavirin or viramidine. In another example, the active agent is interferon.

### General Synthetic Methods

The compounds disclosed herein can be prepared by modifying known methods for forming thiazole compounds and by following the general procedures and examples set forth below. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and P. G. M. Wuts, Protecting Groups in Organic Synthesis, Third Edition, Wiley, New York, 1999, and references cited therein.

If the compounds of this invention contain one or more chiral centers, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

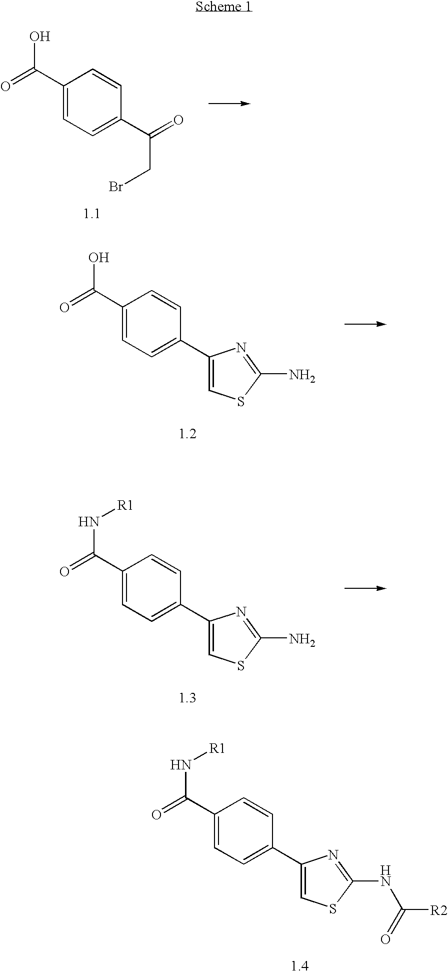

For illustrative purposes, Scheme 1 shows the synthesis of certain thiazole compounds where L¹R¹ is C(O)NHR¹ and L²R² is NHC(O)R² and where L¹, L², R¹, and R² are previously defined. Bromide 1.1 is reacted with thiourea and sodium acetate to give 2-aminothiazole 1.2. Coupling of the acid and amino moieties of 1.2 with the appropriate amine R¹NH₂ or acid R²COOH under amide forming conditions gives amides 1.3 and 1.4. The formation of 1.3 and 1.4 can also involve the use of protecting groups such as amine protecting groups.

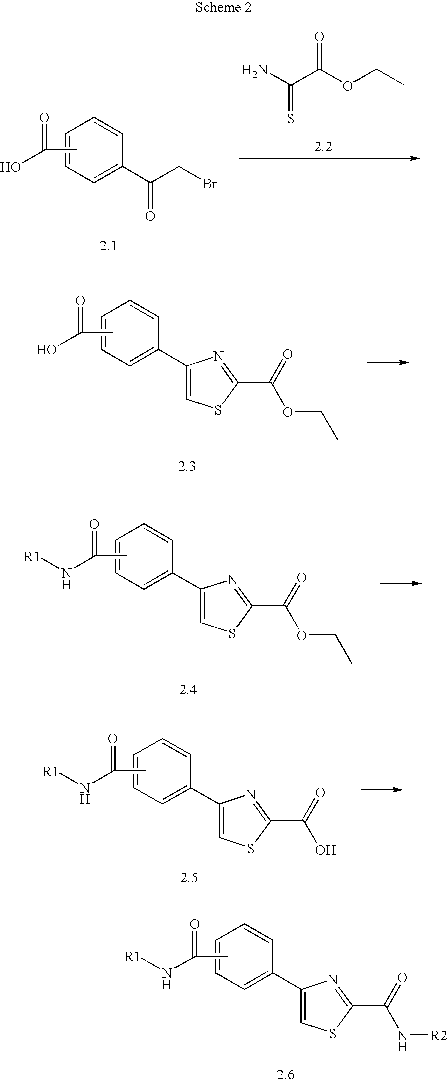

For illustrative purposes, Scheme 2 shows the synthesis of certain thiazole compounds where L¹R¹ is C(O)NHR¹ and L²R² is C(O)NHR² and where L¹, L², R¹, and R² are previously defined. Bromide 2.1 is reacted with amino-thioxo-acetic acid ethyl ester 2.2 under cyclization conditions such as by refluxing in toluene to give thiazole 2.3. Coupling of acid 2.3 with an amine R¹NH₂ under amide forming conditions gives amide 2.4. Hydrolysis of the ester moiety in 2.4 such as by treatment with sodium hydroxide under saponification conditions gives acid 2.5 that is then coupled with R²NH₂ to give the bis-amide 2.6.

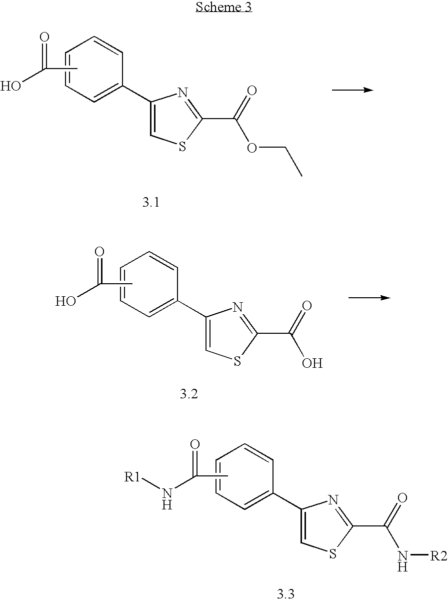

For illustrative purposes, Scheme 3 shows the synthesis of certain thiazole compounds where L¹R¹ is C(O)NHR¹ and L²R² is C(O)NHR² and where L¹, L², R¹, and R² are previously defined and where R¹ and R² are identical. Thiazole ethyl ester 3.1. is hydrolyzed such as treatment with sodium hydroxide under saponification conditions to give the bis-acid 3.2. Coupling of the two acids of 3.2 with an amine R¹NH₂ under amide forming conditions gives amide 3.3.

Suitable amide coupling conditions include the use of coupling reagents. A variety of amide coupling reagents may be used to from the amide bond, including the use of carbodiimides such as N—N′-dicyclohexylcarbodiimide (DCC), N—N′-diisopropylcarbodiimide (DIPCDI), and 1-ethyl-3-(3′-dimethylaminopropyl)carbodiimide (EDCI). The carbodiimides may be used in conjunction with additives such as benzotriazoles 7-aza-1-hydroxybenzotriazole (HOAt), 1-hydroxybenzotriazole (HOBt), and 6-chloro-1-hydroxybenzotriazole (Cl-HOBt).

Amide coupling reagents also include amininum and phosphonium based reagents. Aminium salts include N—[(dimethylamino)-1H-1,2,3-triazolo[4,5-b]pyridine-1-ylmethylene]-N-methylmethanaminium hexafluorophosphate N-oxide (HATU), N-[(1H-benzotriazol-1-yl)(dimethylamino)methylene]-N-methylmethanaminium hexafluorophosphate N-oxide (HBTU), N-[(1H-6-chlorobenzotriazol-1-yl)(dimethylamino)methylene]-N-methylmethanaminium hexafluorophosphate N-oxide (HCTU), N-[(1H-benzotriazol-1-yl)(dimethylamino)methylene]-N-methylmethanaminium tetrafluoroborate N-oxide (TBTU), and N-[(1H-6-chlorobenzotriazol-1-yl)(dimethylamino)methylene]-N-methylmethanaminium tetrafluoroborate N-oxide (TCTU). Phosphonium salts include 7-azabenzotriazol-1-yl-N-oxy-tris(pyrrolidino)phosphonium hexafluorophosphate (PyAOP) and benzotriazol-1-yl-N-oxy-tris(pyrrolidino)phosphonium hexafluorophosphate (PyBOP).

The amide formation step may be conducted in a polar solvent such as dimethylformamide (DMF) and may also include an organic base such as diisopropylethylamine

(DIPEA).

The foregoing and other aspects of the present invention may be better understood in connection with the following representative examples.

### EXAMPLES

In the examples below and the synthetic schemes above, the following abbreviations have the following meanings. If an abbreviation is not defined, it has its generally accepted meaning.
μL=microliters
μm=micromolar
NMR=nuclear magnetic resonance
br=broad
d=doublet
δ=chemical shift
oc=degrees celcius
dd=doublet of doublets
DMEM=Dulbeco's Modified Eagle's Medium
DMF=N,N-dimethylformamide
DMSO=dimethylsulfoxide
DTT=dithiothreotol
EDTA=ethylenediaminetetraacetic acid
ESI=electrospray ionization
EtOH=ethanol
g=gram
h or hr=hours
HCV=hepatitus C virus
HPLC=high performance liquid chromatography
Hz=hertz
IPTG=isopropyl-β-D-thiogalactopyranoside
IU=International Units
IC₅₀=inhibitory concentration at 50% inhibition
J=coupling constant (given in Hz unless otherwise indicated)
m=multiplet
M=molar
M+H⁺=parent mass spectrum peak plus H⁺
mg=milligram
mL=milliliter
mM=millimolar
mmol=millimole
MS=mass spectrum
NaOAc=sodium acetate
nM=nanomolar
ng=nanogram
NTA=nitrilotriacetic acid
NTP=nucleoside triphosphate
PCR=Polymerase chain reaction
ppm=parts per million
psi=pounds per square inch
HPLC=high performance liquid chromatographY
s=Singlet
t=triplet
TC₅₀=Toxic concentration at 50% cell toxicity
Tris=Tris(hydroxymenthyl)aminomethane
UTP=uridine triphosphate

Set forth in the examples below are compounds and intermediates useful for making such compounds. An overview of the synthetic protocols employed to prepare these compounds is set forth above.

### Example 1

### 4-{2-[4-(1,1-Dioxo-thiomorpholin-4-ylmethyl)-benzoylamino]-thiazol-4-yl}-N-(4-morpholin-4-yl-phenyl)-benzamide (Compound 101)

### 4-(2-Amino-thiazol-4-yl)-benzoic acid

2 g (8.2 mmol) of 4-(2-bromo-acetyl)-benzoic acid was combined with 0.625 (8.2 mmol) of thiourea. To this mixture was added NaOAc (0.4 g). This mixture was suspended in 35 mL of EtOH and stirred at room temperature for 2 hours. The mixture was evaporated to dryness and washed using cold diethyl ether. The resulting white powder was used in the next step without further purification.

### 4-(2-Amino-thiazol-4-yl)-N-(4-morpholin-4-yl-phenyl)-benzamide

100 mg (0.45 mmol) of 4-(2-Amino-thiazol-4-yl)-benzoic acid was combined with 171 mg (0.45 mmol) of HATU. This mixture was dissolved in 3 mL of DMF. To this mixture was added DIEA (0.1 mL). Reaction mixture was stirred at room temperature for 40 minutes, at which point 80.2 mg (0.45 mmol) of 4-morpholin-4-yl-phenylamine was added. Reaction mixture was heated at 60° C. overnight. The crude product was purified using reverse phase HPLC.

### 4-{2-[4-(1,1-Dioxo-thiomorpholin-4-ylmethyl)-benzoylamino]-thiazol-4-yl}-N-(4-morpholin-4-yl-phenyl)-benzamide (Compound 101)

100 mg (0.26 mmol) of 4-(2-Amino-thiazol-4-yl)-N-(4-morpholin-4-yl-phenyl)-benzamide was combined with 80.8 mg (0.3 mmol) of 4-(1,1-dioxo-thiomorpholin-4-ylmethyl)-benzoic acid. To this mixture were added 80 mg of EDCI hydrochloride and 60 mg of HOBt. The mixture was dissolved in 3.5 mL of DMF and 0.1 mL of N-methyl morpholine. Reaction mixture was stirred at 50° C. overnight. It was purified using reverse phase HPLC. MS: 632.7 (M+H⁺); H¹ NMR (DMSO-d₆): δ(ppm) 12.80 (s, 1H), 10.09 (s, 1H), 8.06 (m, 6H), 7.88 (s, 1H), 7.62 (d, 2H), 7.52 (d, 2H), 6.93 (d, 2 h), 3.83 (m, 1H), 3.74 (m, 4H), 3.17 (m, 4H), 3.08 (m, 4H), and 2.95 (m, 4H).

### Example 2

### 4-{2-[4-(1,1-Dioxo-thiomorpholin-4-ylmethyl)-benzoylamino]-thiazol-4-yl}-N-cyclopropyl-benzamide (Compound 102)

Prepared using the procedure for compound 101. MS: 511.1 (M+H⁺); ¹H NMR (DMSO-d₆): δ(ppm) 8.23 (d, 2H), 7.92 (m, 4H), 7.70 (m, 3H), 4.19 (s, 2H), 3.34 (m, 8H), 2.95 (m, 1H), 0.76 (m, 2H), and 0.63 (m, 2H).

### Example 3

### 4-[2-(4-Morpholin-4-yl-benzoylamino)-thiazol-4-yl]-N-[4-(1,1-dioxo-thiomorpholin-4-ylmethyl)-phenyl]-benzamide (Compound 103)

Prepared using the procedure for compound 101. MS: 632.7 (M+H); ¹H NMR (Acetone-d₆): δ(ppm) 9.74 (s, 1H), 8.11 (m, 6H), 7.94 (d, 2H), 7.67 (s, 1H), 7.59 (d, 2H), 7.08 (d, 2H), 4.47 (s, 2H), 3.77 (m, 8H), 3.60 (m, 4H), and 3.36 (m, 4H).

### Example 4

### 3-{2-[4-(1,1-Dioxo-thiomorpholin-4-ylmethyl)-benzoylamino]-thiazol-4-yl}-N-(4-morpholin-4-yl-phenyl)-benzamide (Compound 104)

Prepared using the procedure for compound 101 starting from 3-(2-bromo-acetyl)-benzoic acid. MS: 632.7 (M+H⁺); ¹H NMR (Acetone-d₆): δ(ppm) 9.63 (s, 1H), 8.54 (m, 1 h), 8.26 (d, 2H), 8.10 (dt, 1H), 7.92 (d, 1H), 7.80 (m, 4H), 7.68 (s, 1H), 7.55 (t, 1H), 7.20 (m, 2H), 4.35 (s, 2H), 3.88 (m, 4H), 3.53 (m, 4H), 3.44 (m, 4H), and 3.29 (m, 4H).

### Example 5

### 4-[4-(4-Morpholin-4-yl-phenylcarbamoyl)-phenyl]-thiazole-2-carboxylic acid

### [4-(1,1-dioxo-thiomorpholin-4-ylmethyl)-phenyl]-amide (Compound 105)

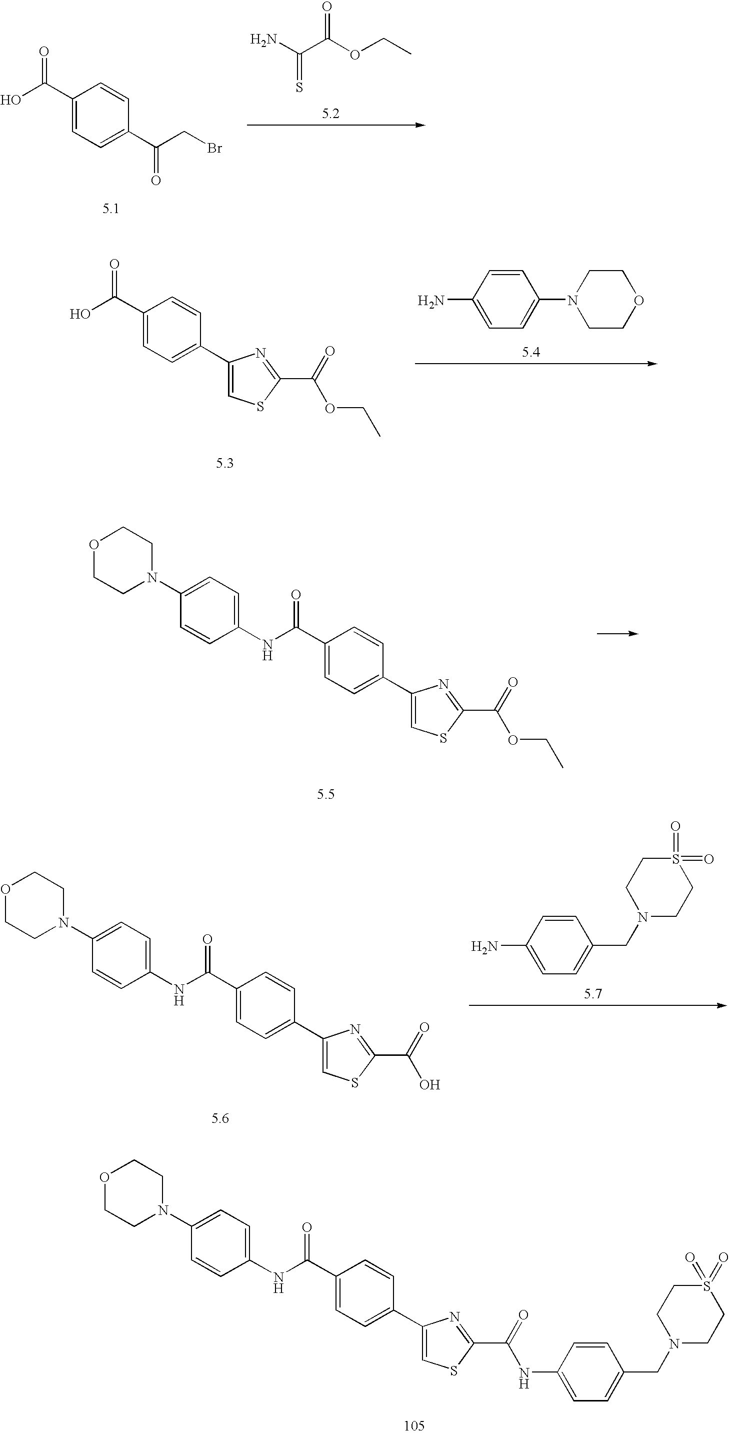

### 4-(4-carboxy-phenyl)-thiazole-2-carboxylic acid ethyl ester

4-(2-Bromo-acetyl)-benzoic acid (1.02 g, 4.2 mmol) and amino-thioxo-acetic acid ethyl ester (0.56 g, 4.2 mmol) were refluxed in toluene (20 mL) for 4 hours. Solids that had precipitated were filtered, washed (15 mL hexanes) and dried to afford 4-(4-carboxy-phenyl)-thiazole-2-carboxylic acid ethyl ester (0.86 g, 74%) as a brown solid. LCMS 278.0 (M+H), 300.0 (M+Na).

### 4-[4-(4-morpholin-4-yl-phenylcarbamoyl)-phenyl]-thiazole-2-carboxylic acid ethyl ester

4-(4-carboxy-phenyl)-thiazole-2-carboxylic acid ethyl ester (0.15 g, 0.54 mmol), 4-morpholin-4-yl-phenylamine (0.097 g, 0.54 mmol) and HBTU (0.24 g, 0.65 mmol) were dissolved in DMF (2 mL). DIPEA (0.19 mL, 1.08 mmol) was added and the mixture stirred for 2 hours. The reaction mixture was added dropwise to cold, aqueous NaHCO₃. The solids that precipitated were filtered, washed (water, 10 mL) and air-dried briefly to afford 4-[4-(4-morpholin-4-yl-phenylcarbamoyl)-phenyl]-thiazole-2-carboxylic acid ethyl ester. LCMS 438.1 (M+H).

### 4-[4-(4-morpholin-4-yl-phenylcarbamoyl)-phenyl]-thiazole-2-carboxylic acid

4-[4-(4-morpholin-4-yl-phenylcarbamoyl)-phenyl]-thiazole-2-carboxylic acid ethyl ester was redissolved in THF-CH₃CN-EtOH (3 mL each), 5% NaOH (2 mL) was added and the mixture stirred for 1 hour. All solvents were evaporated and upon adding 1 N HCl (to acidify), a gray solid precipitated. The solid was filtered, washed (water) and dried to afford crude 4-[4-(4-morpholin-4-yl-phenylcarbamoyl)-phenyl]-thiazole-2-carboxylic acid (0.17 g). LCMS 410.1 (M+H).

### 4-[4-(4-morpholin-4-yl-phenylcarbamoyl)-phenyl]-thiazole-2-carboxylic acid [4-(1,1-dioxo-thiomorpholin-4-ylmethyl)-phenyl]-amide (Compound 105)

4-[4-(4-morpholin-4-yl-phenylcarbamoyl)-phenyl]-thiazole-2-carboxylic acid was converted to the title compound using the amide coupling procedure described above using 4-(1,1-dioxo-thiomorpholin-4-ylmethyl)-phenylamine. After workup, purification (reverse phase HPLC) and lyophilization afforded 0.025 g (9.5%) of 4-[4-(4-morpholin-4-yl-phenylcarbamoyl)-phenyl]-thiazole-2-carboxylic acid [4-(1,1-dioxo-thiomorpholin-4-ylmethyl)-phenyl]-amide.

LCMS 632.2 (M+H). ¹H NMR (DMSO-d₆) δ(ppm) 10.83 (s, 1H), 10.34 (s, 1H), 8.69 (s, 1H), 8.30 (d, 2H), 8.09 (d, 2H), 7.94 (d, 2H), 7.76 (d, 2H), 7.62 (d, 2H), 7.28 (br d, 2H), 4.44 (s, 2H), 3.85 (br m, under the water peak), and 3.23 (br s, 4H).

### Example 6

### 4-{4-[4-(1,1-Dioxo-thiomorpholin-4-ylmethyl)-phenylcarbamoyl]-phenyl}-thiazole-2-carboxylic acid (4-morpholin-4-yl-phenyl)-amide (Compound 106)

Prepared using the procedure for compound 105. LCMS 632.2 (M+H). ¹H NMR (DMSO-d₆) δ(ppm) 10.50 (s, 1H), 10.41 (s, 1H), 8.64 (s, 1H), 8.30 (d, 2H), 8.07 (d, 2H), 7.83 (d, 2H), 7.71 (d, 2H), 7.44 (br s, 2H), 6.97 (d, 2H), 4.12 (br s, 2H), 3.74 (br m, 4H), 3.51 (br m, under the water peak), and 3.10 (br m, 4H).

### Example 7

### 4-[3-(4-Morpholin-4-yl-phenylcarbamoyl)-phenyl]-thiazole-2-carboxylic acid [4-(1,1-dioxo-thiomorpholin-4-ylmethyl)-phenyl]-amide (Compound 107)

Prepared using the procedure for compound 105. LCMS 632.2 (M+H). ¹H NMR (DMSO-d₆) δ(ppm) 10.90 (s, 1H), 10.49 (s, 1H), 8.75 (s, 1H), 8.66 (s, 1H), 8.33 (d, 1H), 7.97 (m, 3H), 7.81 (d, 2H), 7.63 (m, 3H), 7.29 (br s, 2H), 4.45 (s, 2H), 3.83 (br s, 4H), 3.64 (br s under the water peak), and 3.24 (br s, 4H).

### Example 8

### 4-{3-[4-(1,1-Dioxo-thiomorpholin-4-ylmethyl)-phenylcarbamoyl]-phenyl}-thiazole-2-carboxylic acid (4-morpholin-4-yl-phenyl)-amide (Compound 108)

Prepared using the procedure for compound 105. LCMS 632.2 (M+H). ¹H NMR (DMSO-d₆) δ(ppm) 10.61 (s, 1H), 10.58 (s, 1H), 8.68 (s, 1H), 8.62 (s, 1H), 8.36 (d, 1H), 7.91 (m, 3H), 7.73 (d, 2H), 7.65 (t, 1H), 7.56 (d, 2H), 7.03 (d, 2H), 4.40 (s, 2H), 3.64 (br s under the water peak), and 3.08 (br s, 4H).

### Example 9

### 3-[2-(4-morpholin-4-yl-benzoylamino)-thiazol-4-yl]-N-[4-(1,1-dioxo-thiomorpholin-4-ylmethyl)-phenyl]-benzamide (Compound 109)

Prepared using the procedure for compound 101 starting from 3-(2-bromo-acetyl)-benzoic acid. MS: 632.7 (M+H⁺); ¹H NMR (Acetone-d₆): δ(ppm) 9.80 (s, 1H), 8.53 (m, 1H), 8.14 (m, 3H), 7.93 (m, 3H), 7.58 (m, 3H), 7.60 (m, 3H), 7.08 (d, 2H), 7.00 (d, 1H), 4.37 (s, 2H), 3.80 (m, 4H), 3.63 (m, 4H), 3.52 (m, 4H), 3.36 (m, 4H), and 3.30 (m, 1H).

### Example 10

### 4-{4-[4-(1,1-Dioxo-thiomorpholin-4-ylmethyl)-phenylcarbamoyl]-phenyl}-thiazole-2-carboxylic acid [4-(1,1-dioxo-thiomorpholin-4-ylmethyl)-phenyl]-amide (Compound 110)

Prepared using the procedure for compound 105 or 114. LCMS 694.7 (M+H⁺). ¹H NMR (DMSO-d₆) δ(ppm) 10.83 (s, 1H), 10.53 (s, 1H), 8.71 (s, 1H), 8.32 (d, 2H), 8.13 (d, 2H), 7.94 (d, 2H), 7.87 (d, 2H), 7.59 (m, 4H), 4.41 (br s, 4H), 3.56 (br m, under the water peak), 2.89 (s, 1H), and 2.28 (d, 1H).

### Example 11

### 4-[4-(4-Morpholin-4-yl-phenylcarbamoyl)-phenyl]-thiazole-2-carboxylic acid (4-morpholin-4-yl-phenyl)-amide (Compound III)

Prepared using the procedure for compound 105 or 114. LCMS 570.7 (M+H⁺). ¹H NMR (DMSO-d₆) δ(ppm) 10.74 (s, 1H), 10.52 (s, 1H), 8.69 (s, 1H), 8.31 (d, 2H), 8.14 (d, 2H), 7.87 (d, 4H), 7.76 (d, 2H), 7.47 (d, 2H), 3.93 (br d, 8H), and 3.37 (br d, 8H).

### Example 12

### 4-[3-(4-Morpholin-4-yl-phenylcarbamoyl)-phenyl]-thiazole-2-carboxylic acid (4-morpholin-4-yl-phenyl)-amide (Compound 112)

Prepared using the procedure for compound 105 or 114. LCMS 570.7 (M+H⁺). ¹H NMR (DMSO-d₆) δ(ppm) 10.58 (s, 1H), 10.29 (s, 1H), 8.65 (s, 1H), 8.60 (s, 1H), 8.33 (d, 1H), 7.92 (br d, 2H), 7.73 (m, 5H), 7.05 (m, 4H), 3.77 (br m, 8H), and 3.140 (br m, 8H).

### Example 13

### 4-{3-[4-(1,1-Dioxo-thiomorpholin-4-ylmethyl)-phenylcarbamoyl]-phenyl}-thiazole-2-carboxylic acid [4-(1,1-dioxo-thiomorpholin-4-ylmethyl)-phenyl]-amide (Compound 113)

Prepared using the procedure for compound 105 or 114. LCMS 694.2 (M+H⁺). ¹H NMR (DMSO-d₆) δ(ppm) 10.95 (s, 1H), 10.75 (s, 1H), 8.84 (s, 1H), 8.71 (s, 1H), 8.36 (d, 1H), 7.98 (m, 5H), 7.63 (m, 5H), 4.45 (br s, 4H), 3.97 (br d, 8H), and 3.63 (br d, 8H).

### Example 14

### 4-(4-{4-[4-(Propane-1-sulfonyl)-piperazin-1-ylmethyl]-phenylcarbamoyl}-phenyl)-thiazole-2-carboxylic acid {4-[4-(propane-1-sulfonyl)-piperazin-1-ylmethyl]-phenyl}-amide (Compound 114)

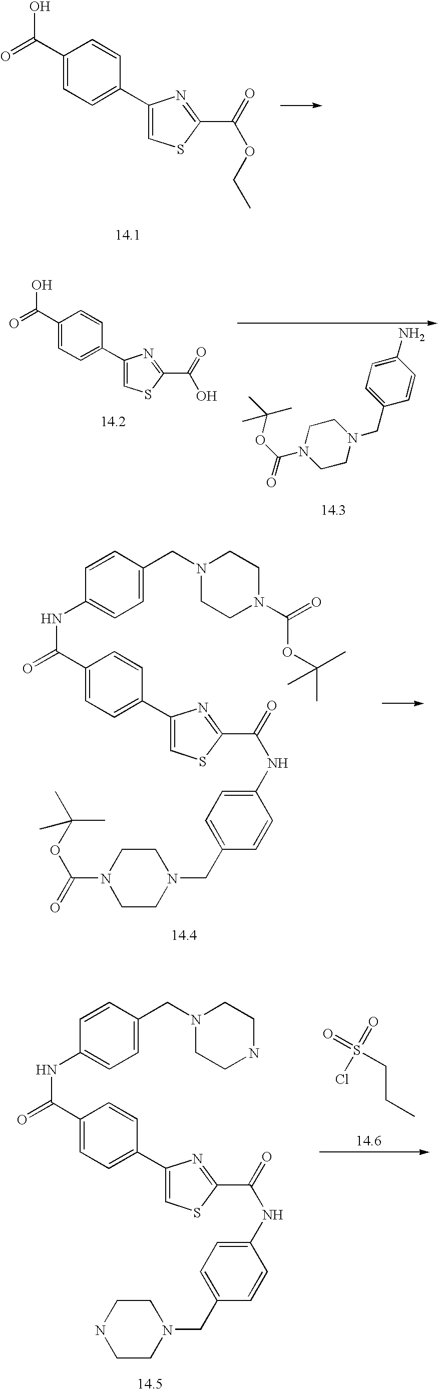

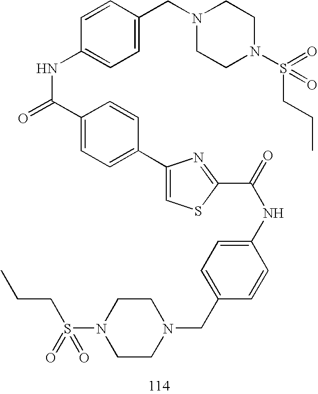

### 4-(4-carboxy-phenyl)-thiazole-2-carboxylic acid

4-(4-Carboxy-phenyl)-thizole-2-carboxylic acid ethyl ester (see Example 5, 250 mg, 0.90 mmol) was suspended in 10 mL of ethanol. To this suspension was added 1M NaOH (5 mL). Reaction mixture was stirred at room temperature for 30 minutes. Most of the ethanol was evaporated under reduced pressure and the resulting solution was acidified using 2M HCl. The precipitate was collected by vacuum filtration, washed using H₂O, and dried to yield the desired material.

### 4-(4-{4-[4-(tert-Butyloxycarbonyl)-piperazin-1-ylmethyl]-phenylcarbamoyl}-phenyl)-thiazole-2-carboxylic acid {4-[4-(tert-butyloxycarbonyl)-piperazin-1-ylmethyl]-phenyl}-amide

4-(4-Carboxy-phenyl)-thiazole-2-carboxylic acid, (100 mg, 0.4 mmol) was combined with HATU (304 mg, 0.8 mmol). This mixture was dissolved in 3 mL of DMF and DIEA (1.0 mmol, 0.17 mL) was added to the solution. Reaction mixture was stirred at room temperature for 30 minutes. To this was added 4-(4-amino-benzyl)-piperazine-1-carboxylic acid tert-butyl ester (233.1 mg, 0.8 mmol). Reaction mixture was heated at 60° C. overnight. The crude was purified using reverse phase HPLC to give the desired product.

### 4-[4-(4-piperazin-1-ylmethyl-phenylcarbamoyl)-phenyl]-thizole-2-carboxylic acid (4-piperazin-1-ylmethyl-phenyl)-amide

4-(4-{4-[4-(tert-Butyloxycarbonyl)-piperazin-1-ylmethyl]-phenylcarbamoyl}-phenyl)-thiazole-2-carboxylic acid {4-[4-(tert-butyloxycarbonyl)-piperazin-1-ylmethyl]-phenyl}-amide (187 mg, 0.23 mmol) was suspended in 10 mL of CH₂Cl₂. To this suspension was added 10 mL of TFA. Reaction mixture was stirred at room temperature for 1 hour and then evaporated to yield the desired product as the TFA salt.

### 4-(4-{4-[4-(Propane-1-sulfonyl)-piperazin-1-ylmethyl]-phenylcarbamoyl}-phenyl)-thiazole-2-carboxylic acid {4-[4-(propane-1-sulfonyl)-piperazin-1-ylmethyl]-phenyl}-amide (compound 114)

4-[4-(4-piperazin-1-ylmethyl-phenylcarbamoyl)-phenyl]-thizole-2-carboxylic acid (4-piperazin-1-ylmethyl-phenyl)-amide (50 mg, 0.084 mmol) was dissolved in 4 mL of CH₂Cl₂. To this solution was added triethylamine (0.21 mmol, 0.03 mL), followed by propane-1-sulfonyl chloride (0.21 mmol, 0.024 mL). Reaction mixture was stirred at room temperature for 1 hour. It was diluted with additional amounts of CH₂Cl₂ and washed using H₂O. Organic phase was isolated, dried, and evaporated. The resulting crude mixture was purified using reverse phase HPLC. MS: 809.7 (M+H⁺); H¹ NMR (DMSO-d₆): δ(ppm) 10.83 (s, 1H), 10.52 (s, 1H), 8.70 (s, 1H), 8.31 (d, 2H), 8.10 (d, 2H), 7.96 (d, 2H), 7.87 (d, 2H), 7.48 (q, 4H), 4.33 (br s, 4H), 3.76 (m, 8H), 3.11 (t, 12H), 1.66 (m, 4H), and 0.98 (t, 6H).

### Example 15

### 4-(3-{4-[4-(Propane-1-sulfonyl)-piperazin-1-ylmethyl]-phenylcarbamoyl}-phenyl)-thiazole-2-carboxylic acid {4-[4-(propane-1-sulfonyl)-piperazin-1-ylmethyl]-phenyl}-amide (Compound 115)

Prepared following the procedure described for compound 114. MS: 808.3 (M+H⁺); H¹ NMR (DMSO-d₆): δ(ppm) 10.85 (s, 1H), 10.58 (s, 1H), 8.63 (s, 1H), 8.61 (br s, 1H), 8.37 (d, 1H), 7.90 (m, 5H), 7.67 (t, 1H), 7.48 (t, 4H), 4.33 (br s, 4H), 3.76 (m, 8H), 3.12 (t, 12H), 1.68 (m, 4H), and 0.98 (t, 6H).

### Example 16

### 4-(3-{4-[4-(Propane-1-sulfonyl)-piperazin-1-ylmethyl]-phenylcarbamoyl}-phenyl)-thiazole-2-carboxylic acid (4-morpholin-4-yl-phenyl)-amide (Compound 116)

Prepared following the procedure described for compound 105. MS: 689.3 (M+H⁺); H¹ NMR (Acetone-d₆): δ(ppm) 9.91 (s, 1H), 8.59 (br s, 1H), 8.12 (m, 4H), 7.97 (m, 3H), 7.62 (m, 4H), 7.06 (d, 2H), 4.45 (s, 2H), 3.81 (t, 2H), 3.34-3.26 (m, 8H), 3.06 (m, 2H), 2.73 (m, 2H), 1.79 (m, 4H), 1.36 (m, 2H), and 1.02 (m, 3H)

### Administration and Pharmaceutical Composition

The present invention provides novel compounds possessing antiviral activity, including Flaviviridae family viruses such as hepatitis C virus. The compounds of this invention inhibit viral replication by inhibiting the enzymes involved in replication, including RNA dependent RNA polymerase. They may also inhibit other enzymes utilized in the activity or proliferation of Flaviviridae viruses.

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors. The drug can be administered more than once a day, preferably once or twice a day.

Therapeutically effective amounts of compounds of the present invention may range from approximately 0.01 to 50 mg per kilogram body weight of the recipient per day; preferably about 0.01-25 mg/kg/day, more preferably from about 0.1 to 10 mg/kg/day. Thus, for administration to a 70 kg person, the dosage range would most preferably be about 7-70 mg per day.

This invention is not limited to any particular composition or pharmaceutical carrier, as such may vary. In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of this invention is inhalation.

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of the present invention in combination with at least one pharmaceutically acceptable excipient. Acceptable excipients are non-toxic, aid administration, and do not adversely affect the therapeutic benefit of the claimed compounds. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of a compound of the present invention based on the total formulation, with the balance being one or more suitable pharmaceutical excipients. Preferably, the compound is present at a level of about 1-80 wt %. Representative pharmaceutical formulations are described in the Formulation Examples section below.

Additionally, the present invention is directed to a pharmaceutical composition comprising a therapeutically effective amount of a compound of the present invention in combination with a therapeutically effective amount of another active agent against RNA-dependent RNA virus and, in particular, against HCV. Agents active against HCV include, but are not limited to, ribavirin, levovirin, viramidine, thymosin alpha-1, an inhibitor of HCV NS3 serine protease, or an inhibitor of inosine monophosphate dehydrognease, interferon-α, pegylated interferon-α (peginterferon-α), a combination of interferon-α and ribavirin, a combination of peginterferon-α and ribavirin, a combination of interferon-α and levovirin, and a combination of peginterferon-α and levovirin. Interferon-α includes, but is not limited to, recombinant interferon-α2a (such as ROFERON interferon available from Hoffman-LaRoche, Nutley, N.J.), interferon-α2b (such as Intron-A interferon available from Schering Corp., Kenilworth, N.J., USA), a consensus interferon, and a purified interferon-α product. For a discussion of ribavirin and its activity against HCV, see J. O, Saunders and S. A. Raybuck, “Inosine Monophosphate Dehydrogenase Consideration of Structure, Kinetics and Therapeutic Potential,” Ann. Rep. Med. Chem., 35:201-210 (2000).

The agents active against hepatitis C virus also include agents that inhibit HCV proteases, HCV polymerase, HCV helicase, HCV NS4B protein, HCV entry, HCV assembly, HCV egress, HCV NS5A protein, and inosine 5′-monophosphate dehydrogenase. Other agents include nucleoside analogs for the treatment of an HCV infection. Still other compounds include those disclosed in WO 2004/014313 and WO 2004/014852 and in the references cited therein. The patent applications WO 2004/014313 and WO 2004/014852 are hereby incorporated by references in their entirety.

Specific antiviral agents include Omega IFN (BioMedicines Inc.), BILN-2061 (Boehringer Ingelheim), Summetrel (Endo Pharmaceuticals Holdings Inc.), Roferon A (F. Hoffman-La Roche), Pegasys (F. Hoffman-La Roche), Pegasys/Ribaravin (F. Hoffman-La Roche), CellCept (F. Hoffman-La Roche), Wellferon (GlaxoSmithKline), Albuferon-α (Human Genome Sciences Inc.), Levovirin (ICN Pharmaceuticals), IDN-6556 (Idun Pharmaceuticals), IP-501 (Indevus Pharmaceuticals), Actimmune (InterMune Inc.), Infergen A (InterMune Inc.), ISIS14803 (ISIS Pharamceuticals Inc.), JTK-003 (Japan Tobacco Inc.), Pegasys/Ceplene (Maxim Pharmaceuticals), Ceplene (Maxim Pharmaceuticals), Civacir (Nabi Biopharmaceuticals Inc.), Intron A/Zadaxin (RegeneRx), Levovirin (Ribapharm Inc.), Viramidine (Ribapharm Inc.), Heptazyme (Ribozyme Pharmaceuticals), Intron A (Schering-Plough), PEG-Intron (Schering-Plough), Rebetron (Schering-Plough), Ribavirin (Schering-Plough), PEG-Intron/Ribavirin (Schering-Plough), Zadazim (SciClone), Rebif (Serono), IFN-β/EMZ701 (Transition Therapeutics), T67 (Tularik Inc.), VX-497 (Vertex Pharmaceuticals Inc.), VX-950/LY-570310 (Vertex Pharmaceuticals Inc.), Omniferon (Viragen Inc.), XTL-002 (XTL Biopharmaceuticals), SCH 503034 (Schering-Plough), isatoribine and its prodrugs ANA971 and ANA975 (Anadys), R1479 (Roche Biosciences), Valopicitabine (Idenix), NIM811 (Novartis), and Actilon (Coley Pharmaceuticals).

In some embodiments, the compositions and methods of the present invention contain a compound of the invention and interferon. In some aspects, the interferon is selected from the group consisting of interferon alpha 2B, pegylated interferon alpha, consensus interferon, interferon alpha 2A, and lymphoblastiod interferon tau.

In other embodiments the compositions and methods of the present invention contain a compound of the invention and a compound having anti-HCV activity is selected from the group consisting of interleukin 2, interleukin 6, interleukin 12, a compound that enhances the development of a type 1 helper T cell response, interfering RNA, anti-sense RNA, Imiqimod, ribavirin, an inosine 5′monophospate dehydrogenase inhibitor, amantadine, and rimantadine.

In still other embodiments, the compound having anti-HCV activity is Ribavirin, levovirin, viramidine, thymosin alpha-1, an inhibitor of NS3 serine protease, and inhibitor of inosine monophosphate dehydrogenase, interferon-alpha, or pegylated interferon-alpha alone or in combination with Ribavirin or viramidine.

In another embodiments, the compound having anti-HCV activity is said agent active against HCV is interferon-alpha or pegylated interferon-alpha alone or in combination with Ribavirin or viramidine.

### Biological Examples

### Example 1

### Anti-Hepatitis C Activity

Compounds can exhibit anti-hepatitis C activity by inhibiting HCV polymerase, by inhibiting other enzymes needed in the replication cycle, or by other pathways. A number of assays have been published to assess these activities. A general method that assesses the gross increase of HCV virus in culture was disclosed in U.S. Pat. No. 5,738,985 to Miles et al. In vitro assays have been reported in Ferrari et al. Jnl. of Vir., 73:1649-1654, 1999; Ishii et al., Hepatology, 29:1227-1235, 1999; Lohmann et al., Jnl of Bio. Chem., 274:10807-10815, 1999; and Yamashita et al., Jnl. of Bio. Chem., 273:15479-15486, 1998.

WO 97/12033, filed on Sep. 27, 1996, by Emory University, listing C. Hagedorn and A. Reinoldus as inventors, which claims priority to U.S. Provisional Patent Application. Ser. No. 60/004,383, filed on September 1995, described an HCV polymerase assay that can be used to evaluate the activity of the of the compounds described herein. Another HCV polymerase assay has been reported by Bartholomeusz, et al., Hepatitis C Virus (HCV) RNA polymerase assay using cloned HCV non-structural proteins; Antiviral Therapy 1996:1(Supp 4) 18-24.

Screens that measure reductions in kinase activity from HCV drugs were disclosed in U.S. Pat. No. 6,030,785, to Katze et al., U.S. Pat. No. 6,228,576, Delvecchio, and U.S. Pat. No. 5,759,795 to Jubin et al. Screens that measure the protease inhibiting activity of proposed HCV drugs were disclosed in U.S. Pat. No. 5,861,267 to Su et al., U.S. Pat. No. 5,739,002 to De Francesco et al., and U.S. Pat. No. 5,597,691 to Houghton et al.

### Example 2

### Replicon Assay

A cell line, ET (Huh-lucubineo-ET) is used for screening of compounds for inhibiting HCV RNA dependent RNA polymerase. The ET cell line is stably transfected with RNA transcripts harboring a I₃₈₉luc-ubi-neo/NS3-3′/ET; replicon with firefly luciferase-ubiquitin-neomycin phosphotransferase fusion protein and EMCV-IRES driven NS3-5B polyprotein containing the cell culture adaptive mutations (E1202G; T12801; K1846T) (Krieger at al, 2001 and unpublished). The ET cells are grown in DMEM (Dulbeco's Modified Eagle's Medium), supplemented with 10% fetal calf serum, 2 mM Glutamine, Penicillin (100 IU/mL)/Streptomycin (100 μg/mL), 1× nonessential amino acids, and 250 μg/mL G418 (“Geneticin”). They are all available through Life Technologies (Bethesda, Md.). The cells are plated at 0.5-1.0×10⁴ cells/well in the 96 well plates and incubated for 24 hrs before adding test compound. The compounds are added to the cells to achieve a final concentration of 0.1 nM to 50 μm and a final DMSO (dimethylsulfoxide) concentration of 0.5%. Luciferase activity is measured 48-72 hours later by adding a lysis buffer and the substrate (Catalog number Glo-lysis buffer E2661 and Bright-Glo luciferase system E2620 Promega, Madison, Wis.). Cells should not be too confluent during the assay. Percent inhibition of replication data is plotted relative to no compound control. Under the same condition, cytotoxicity of the compounds are determined using cell proliferation reagent, WST-1(Roche, Germany). The compounds showing antiviral activities, but no significant cytotoxicities are chosen to determine IC₅₀ and TC₅₀. For these determinations, a 10 point, 2-fold serial dilution for each compound was used, which spans a concentration range of 1000 fold. IC₅₀ and TC₅₀ values were calculated by fitting % inhibition at each concentration to the following equation:

% inhibition=100%/[(IC₅₀/[I])^{b}+1]

where b is Hill's coefficient.

Table 2 displays the percent inhibition of replication at 10 μM concentration of compound. Compounds which are not shown in the table are contemplated to have inhibitory activities when tested at higher concentrations.

| Cmpd. # | % Inhib. at 10 μM |
|---|---|
| 102 | 47.4 |
| 104 | 95.8 |
| 107 | 36.5 |
| 108 | 63.1 |
| 109 | 99.8 |
| 110 | 99.9 |
| 111 | 34.2 |
| 112 | 98.5 |
| 113 | 89.5 |
| 114 | 98.0 |
| 115 | 100.0 |
| 116 | 99.5 |

### Formulation Examples

The following are representative pharmaceutical formulations containing a compound of Formula (I).

### Formulation Example 1

### Tablet Formulation

The following ingredients are mixed intimately and pressed into single scored tablets.

| | Quantity per |
|---|---|
| Ingredient | tablet, mg |
| compound | 400 |
| cornstarch | 50 |
| croscarmellose sodium | 25 |
| lactose | 120 |
| magnesium stearate | 5 |

### Formulation Example 2

### Capsule Formulation

The following ingredients are mixed intimately and loaded into a hard-shell gelatin capsule.

| | Quantity per |
|---|---|
| Ingredient | capsule, mg |
| compound | 200 |
| lactose, spray-dried | 148 |
| magnesium stearate | 2 |

### Formulation Example 3

### Suspension Formulation

The following ingredients are mixed to form a suspension for oral administration.

| Ingredient | Amount | |
|---|---|---|
| compound | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |
| distilled water | q.s. to 100 | mL |

### Formulation Example 4

### Injectable Formulation

The following ingredients are mixed to form an injectable formulation.

| Ingredient | Amount |
|---|---|
| compound | 0.2 mg-20 mg |
| sodium acetate buffer solution, 0.4 M | 2.0 mL |
| HCl (1N) or NaOH (1N) | q.s. to suitable pH |
| water (distilled, sterile) | q.s. to 20 mL |

### Formulation Example 5

### Suppository Formulation

A suppository of total weight 2.5 g is prepared by mixing the compound with Witepsol® H-15 (triglycerides of saturated vegetable fatty acid; Riches-Nelson, Inc., New York), and has the following composition:

| Ingredient | Amount |
|---|---|
| compound | 500 mg |
| Witepsol ® H-15 | balance |

What is claimed is:

1. A compound that is Formula (I): 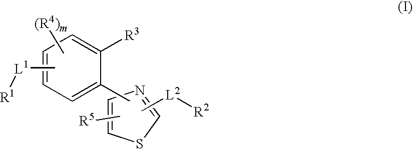
or a pharmaceutically acceptable salt or solvate thereof,
wherein:
L¹ and L² are independently selected from the group consisting of —C(O)NR^{a}-T-, —NR^{a}—C(O)-T-, —NR^{a}C(O)NR^{a}-T-, —NR^{a}C(O)C(O)-T-, —NR^{a}C(O)O-T-, —CH₂NR^{a}-T-, —NR^{a}CH₂-T-, —S(O)₂NH-T-, —NHS(O)₂-T-, and —CH₂NHS(O)₂-T-, where T is attached to R¹ or R² and is independently a covalent bond or C₁₋₃alkylene;
R^{a} is independently hydrogen or alkyl;
R¹ and R² are independently selected from the group consisting of alkyl, substituted alkyl, cycloalkyl, substituted cycloalkyl, heterocyclic, substituted heterocyclic, aryl, substituted aryl, heteroaryl, and substituted heteroaryl;
R³ and R⁵ are independently selected from the group consisting of hydrogen, halo, alkyl, substituted alkyl, alkenyl, substituted alkenyl, alkynyl, substituted alkynyl, hydroxy, alkoxy, substituted alkoxy, amino, substituted amino, azido, cycloalkyl, substituted cycloalkyl, and cyano;
each R⁴ is independently selected from the group consisting of halo, alkyl, substituted alkyl, alkoxy, substituted alkoxy, and hydroxy; and
m is 0, 1, 2 or 3.

2. A compound of claim 1 that is Formula (Ia) or (Ib) 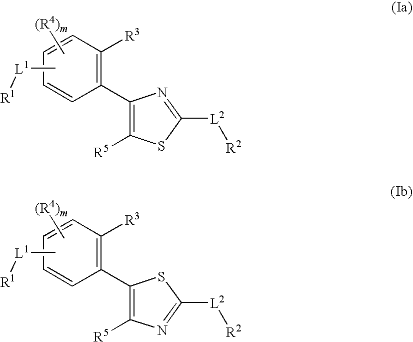
or a pharmaceutically acceptable salt or solvate thereof wherein L¹, L², R¹, R², R⁴, R⁴, R⁵, and m are as defined for Formula (I).

3. A compound of claim 1 that is Formula (Ic) or (Id) 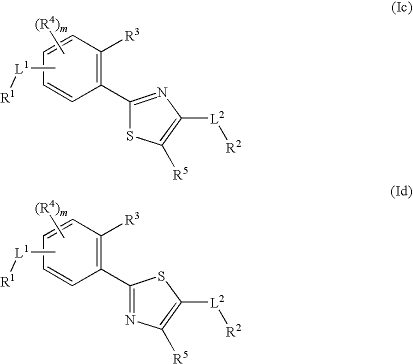
or a pharmaceutically acceptable salt or solvate thereof wherein L¹, L², R¹, R², R³, R⁴, R⁵, and m are as defined for Formula (I).

4. A compound of claim 1 wherein L¹ is para to the thiazole ring.

5. A compound of claim 1 wherein L¹ is para to R³.

6. A compound of claim 1 wherein L¹ and L² are independently —C(O)NR^{a}-T- or —NR^{a}—C(O)-T-.

7. A compound of claim 1 wherein R³ is hydrogen, halo, alkoxy, or haloalkoxy.

8. A compound of claim 1 wherein R⁴ is alkyl.

9. A compound of claim 1 wherein R¹ and R² are independently selected from the group consisting of cycloalkyl, substituted cycloalkyl, heterocyclic, substituted heterocyclic, aryl, substituted aryl, heteroaryl, and substituted heteroaryl.

10. A compound of claim 1 wherein R¹ and R² are independently -G-(Z)ₚ, wherein G is selected from the group consisting of alkyl, alkoxy, amino, acyl, 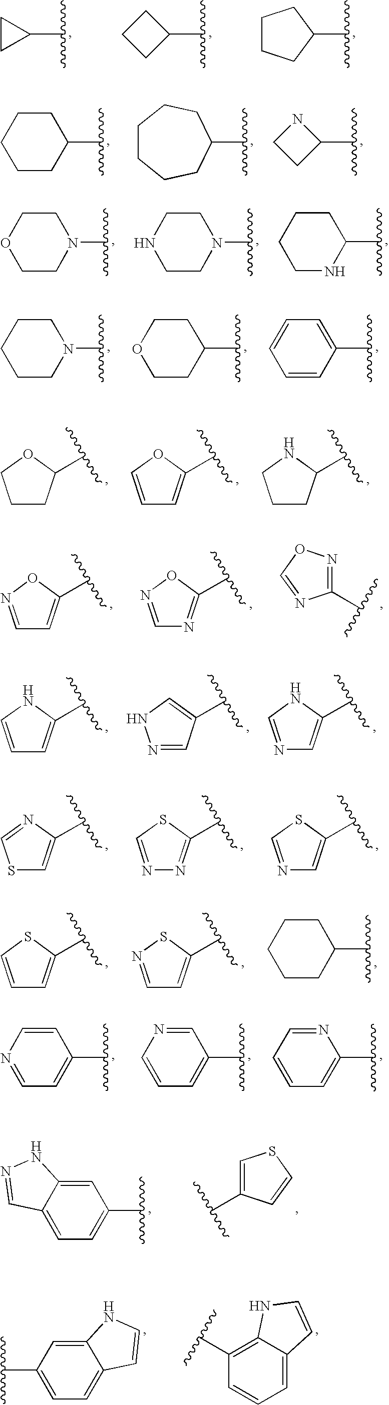 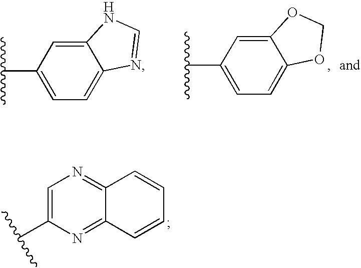
Z is independently selected from the group consisting of alkyl, substituted alkyl, amino, substituted amino, acyl, cyano, halo, hydroxy, alkoxy, substituted alkoxy, cycloalkyl, substituted cycloalkyl, heterocyclic, substituted heterocyclic, aryl, substituted aryl, heteroaryl, substituted heteroaryl, aminocarbonyl, and acylamino; and
p is 0, 1, 2, or 3.

11. A compound of claim 10 wherein p is 0.

12. A compound of claim 10, wherein Z is independently selected from the group consisting of —F, Cl, —Br, —CH₃, —CF₃, —OMe, —OCF₃, —CN, carboxyl ester, —NHC(O)CH₃, 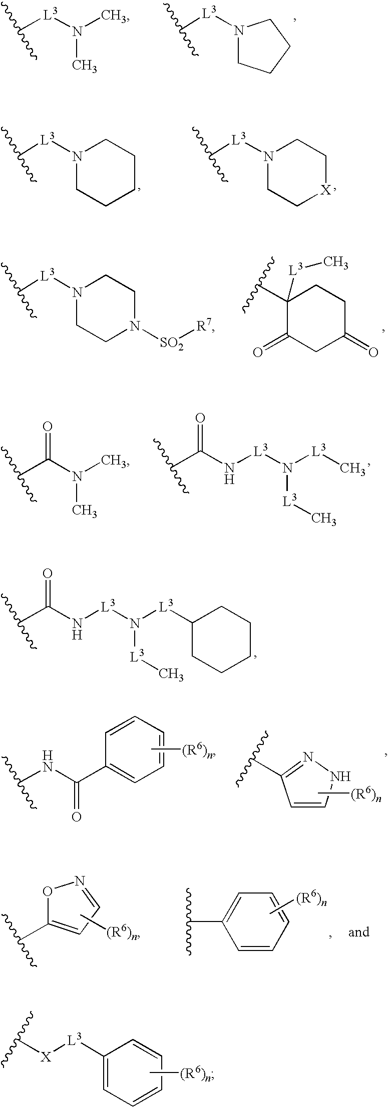
wherein
each R⁶ is independently selected from the group consisting of alkyl, substituted alkyl, alkoxy, substituted alkoxy, and halo;
R⁷ is alkyl, substituted alkyl, cyloalkyl, substituted cycloalkyl, amino, or substituted amino;
R⁸ is hydrogen, alkyl, or substituted alkyl,
L³ is a covalent bond or is C₁₋₃alkylene;
X is selected from the group consisting of O, S, S(O), S(O)₂, and NR⁸; and
n is 0, 1, 2, or 3.

13. A compound of claim 10, wherein G is phenyl.

14. A compound of claim 13, wherein Z is independently selected from the group consisting of —F, —Cl, —Br, —CH₃, —CF₃, CN, hydroxy, alkoxy, 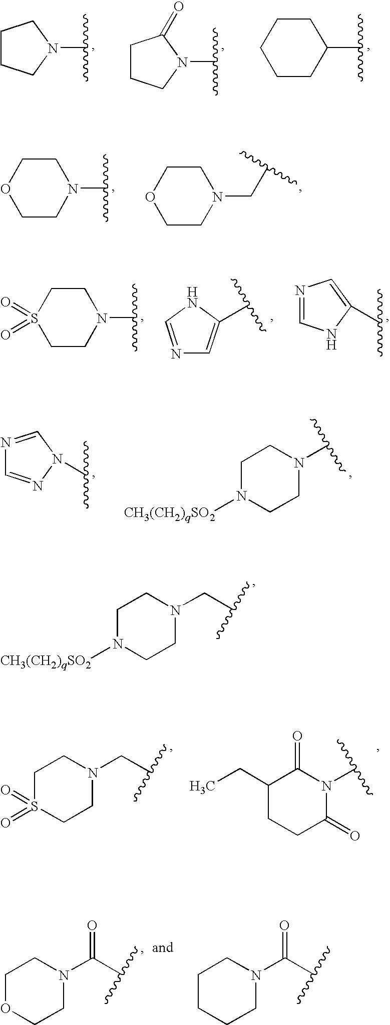
where q is 0, 1, 2, or 3.

15. A compound of claim 10, wherein R¹ is selected from the group consisting of 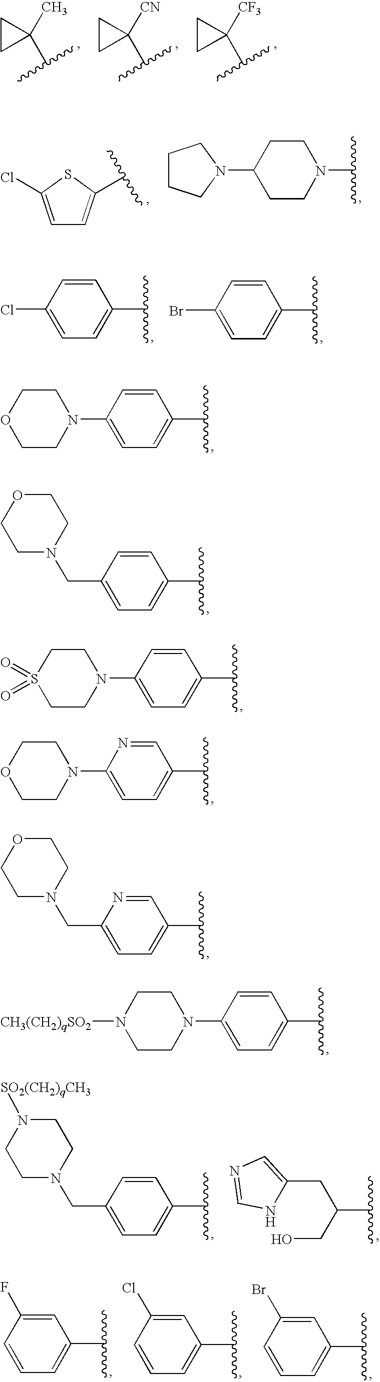 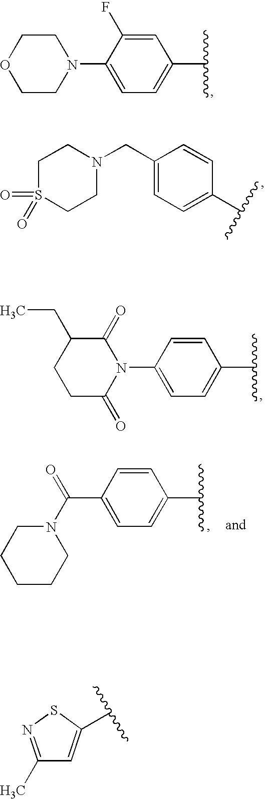
where q is 0, 1, 2, or 3.

16. A compound of claim 10, wherein R² is selected from the group consisting of 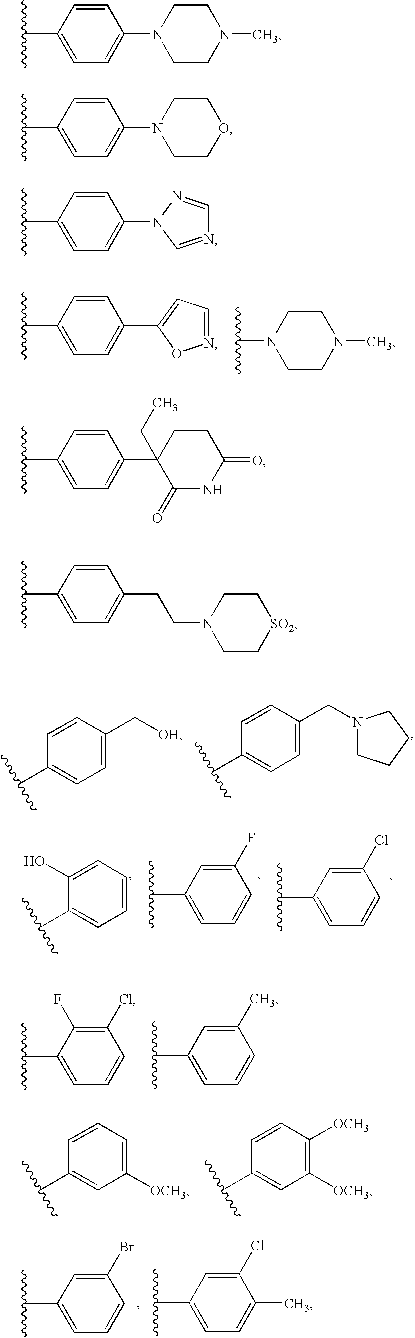 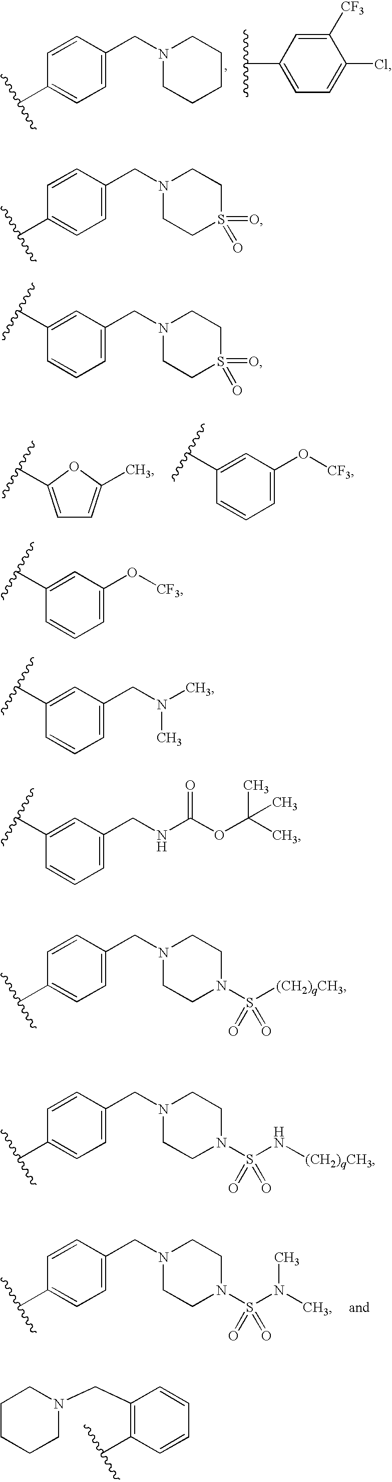
and where q is 0, 1, 2, or 3.

17. A compound of claim 1 that is a compound selected from the Table below or a pharmaceutically acceptable salt or solvate thereof:

| Cmpd. # | Structure | Name |
|---|---|---|
| 101 | 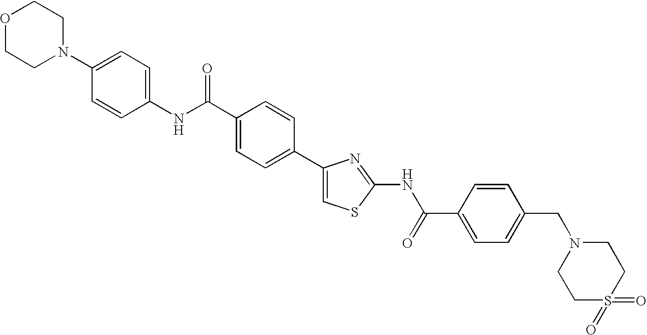 | 4-{2-[4-(1,1-Dioxo- thiomorpholin-4- ylmethyl)- benzoylamino]- thiazol-4-yl}-N-(4- morpholin-4-yl- phenyl)-benzamide |
| 102 | 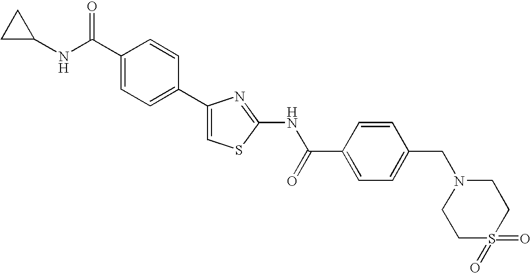 | 4-{2-[4-(1,1-Dioxo- thiomorpholin-4- ylmethyl)- benzoylamino]- thiazol-4-yl}-N- cyclopropyl- benzamide |
| 103 | 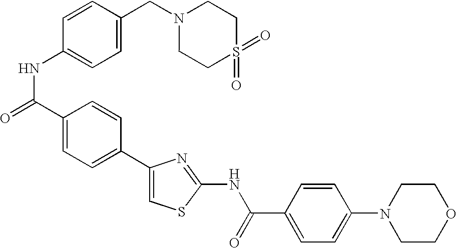 | 4-[2-(4-Morpholin-4- yl-benzoylamino)- thiazol-4-yl]-N-[4- (1,1-dioxo- thiomorpholin-4- ylmethyl)-phenyl]- benzamide |
| 104 | 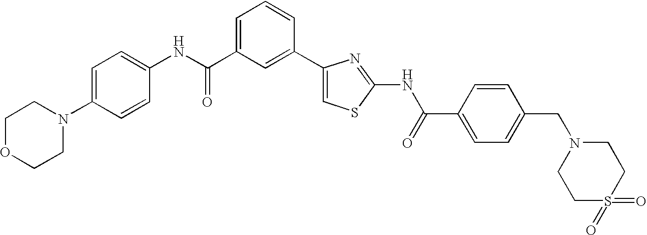 | 3-{2-[4-(1,1-Dioxo- thiomorpholin-4- ylmethyl)- benzoylamino]- thiazol-4-yl}-N-(4- morpholin-4-yl- phenyl)-benzamide |
| 105 | 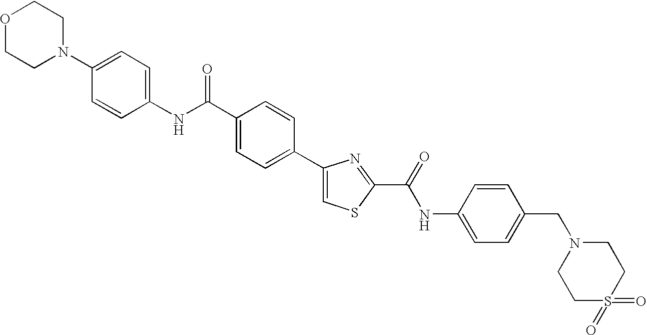 | 4-[4-(4-Morpholin-4- yl-phenylcarbamoyl)- phenyl]-thiazole-2- carboxylic acid [4- (1,1-dioxo- thiomorpholin-4- ylmethyl)-phenyl]- amide |
| 106 | 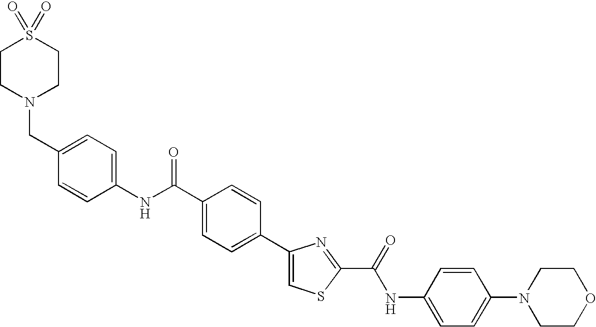 | 4-{4-[4-(1,1-Dioxo- thiomorpholin-4- ylmethyl)- phenylcarbamoyl]- phenyl}-thiazole-2- carboxylic acid (4- morpholin-4-yl- phenyl)-amide |
| 107 | 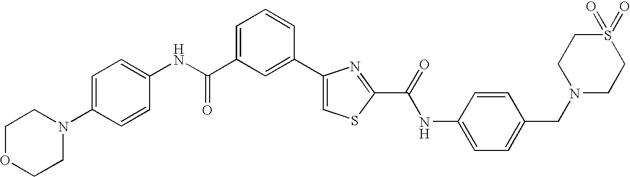 | 4-[3-(4-Morpholin-4- yl-phenylcarbamoyl)- phenyl]-thiazole-2- carboxylic acid [4- (1,1-dioxo- thiomorpholin-4- ylmethyl)-phenyl]- amide |
| 108 | 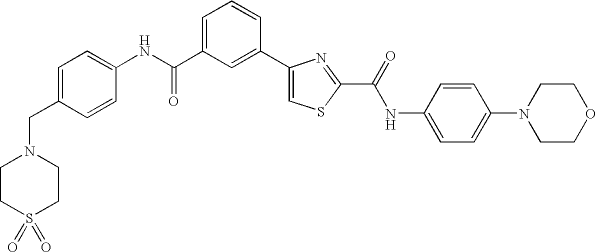 | 4-{3-[4-(1,1-Dioxo- thiomorpholin-4- ylmethyl)- phenylcarbamoyl]- phenyl}-thiazole-2- carboxylic acid (4- morpholin-4-yl- phenyl)-amide |
| 109 | 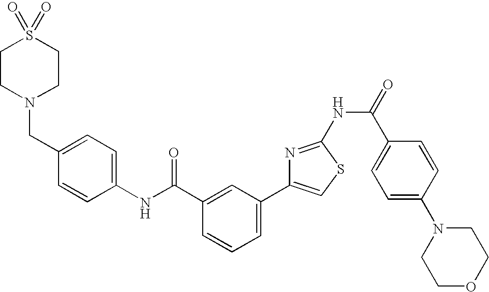 | 3-[2-(4-Morpholin-4- yl-benzoylamino)- thiazol-4-yl]-N-[4- (1,1-dioxo- thiomorpholin-4- ylmethyl)-phenyl]- benzamide |
| 110 | 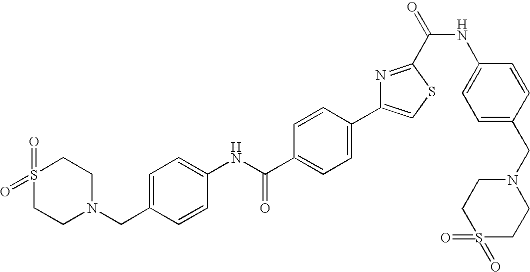 | 4-{4-[4-(1,1-Dioxo- thiomorpholin-4- ylmethyl)- phenylcarbamoyl]- phenyl}-thiazole-2- carboxylic acid [4- (1,1-dioxo- thiomorpholin-4- ylmethyl)-phenyl]- amide |
| 111 | 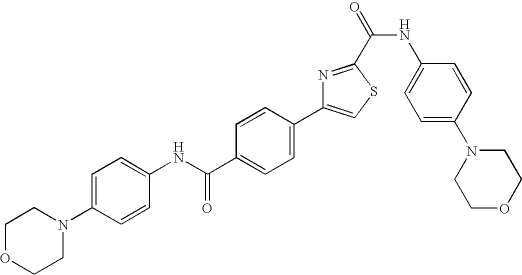 | 4-[4-(4-Morpholin-4- yl-phenylcarbamoyl)- phenyl]-thiazole-2- carboxylic acid (4- morpholin-4-yl- phenyl)-amide |
| 112 | 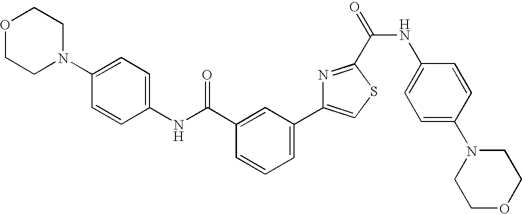 | 4-[3-(4-Morpholin-4- yl-phenylcarbamoyl)- phenyl]-thiazole-2- carboxylic acid (4- morpholin-4-yl- phenyl)-amide |
| 113 | 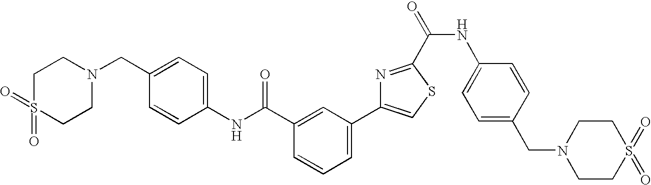 | 4-{3-[4-(1,1-Dioxo- thiomorpholin-4- ylmethyl)- phenylcarbamoyl]- phenyl}-thiazole-2- carboxylic acid [4- (1,1-dioxo- thiomorpholin-4- ylmethyl)-phenyl]- amide |
| 114 | 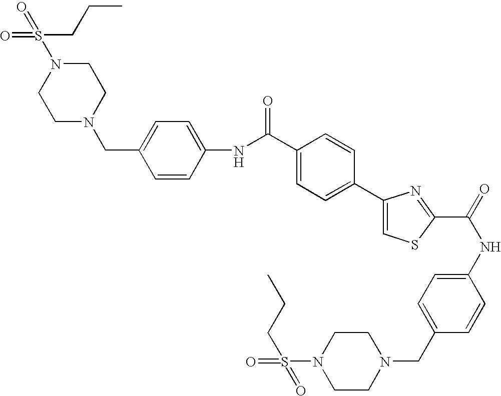 | 4-(4-{4-[4-(Propane- 1-sulfonyl)- piperazin-1- ylmethyl]- phenylcarbamoyl}- phenyl)-thiazole-2- carboxylic acid {4- [4-(propane-1- sulfonyl)-piperazin- 1-ylmethyl]-phenyl}- amide |
| 115 | 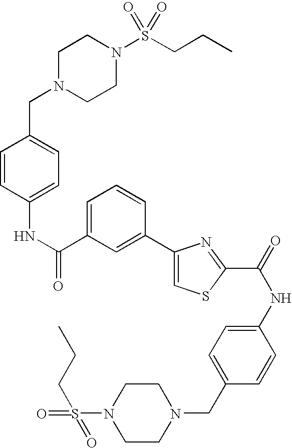 | 4-(3-{4-[4-(Propane- 1-sulfonyl)- piperazin-1- ylmethyl]- phenylcarbamoyl}- phenyl)-thiazole-2- carboxylic acid {4- [4-(propane-1- sulfonyl)-piperazin- 1-ylmethyl]-phenyl}- amide |
| 116 | 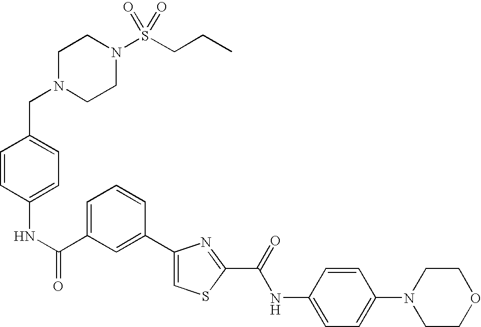 | 4-(3-{4-[4-(Propane- 1-sulfonyl)- piperazin-1- ylmethyl]- phenylcarbamoyl}- phenyl)-thiazole-2- carboxylic acid (4- morpholin-4-yl- phenyl)-amide |

18. A pharmaceutical composition comprising a pharmaceutically acceptable carrier and a therapeutically effective amount of a compound of claim 1.

19. A method for treating a viral infection in a patient mediated at least in part by a virus in the Flaviviridae family of viruses which method comprises administering to the patient a compound of claim 1.

20. The method of claim 19 wherein said viral infection is a hepatitis C mediated viral infection.

21. The method of claim 19 in combination with the administration of a therapeutically effective amount of one or more agents active against hepatitis C virus.

22. The method of claim 21 wherein said agent active against hepatitis C virus is an inhibitor of HCV proteases, HCV polymerase, HCV helicase, HCV NS4B protein, HCV entry, HCV assembly, HCV egress, HCV NS5A protein, or inosine 5′-monophosphate dehydrogenase.

23. The method of claim 21 wherein said agent active against hepatitis C virus is interferon.
